(19) United States
(12) Patent Application Publication
(10) Pub. No.: US 2024/0240239 A1
(43) Pub. Date: Jul. 18, 2024

(54) METHOD FOR TESTING PRESENCE OR LEVEL OF ONE OR MORE TARGET NUCLEIC ACIDS IN SAMPLE

(71) Applicant: XIAMEN SEERNA BIOSCIENCE CO., LTD., Xiamen, Fujian (CN)

(72) Inventors: Ling LIU, Xiamen, Fujian (CN); Lihua HUANG, Xiamen, Fujian (CN); Rongqin KE, Xiamen, Fujian (CN)

(21) Appl. No.: 18/560,358

(22) PCT Filed: Mar. 22, 2022
(86) PCT No.: PCT/CN22/82496

§ 371 Date: Nov. 10, 2023

(30) Foreign Application Priority Data
May 13, 2021 (CN) 202110521332.4

(51) Int. Cl. C12Q 1/6825; C12Q 1/25; C12Q 1/48; C12Q 1/6844; C12Q 1/6876

(52) U.S. Cl. CPC C12Q 1/6825; C12Q 1/25; C12Q 1/485; C12Q 1/6844; C12Q 1/6876; G01N 2333/9015; G01N 2333/9126

(57) ABSTRACT

The present invention relates to a method for testing the presence or level of one or more target nucleic acids in a sample, and further relates to a probe set and a kit comprising one or more probe sets.

### TECHNICAL FIELD

The present invention relates to the field of biotechnology, and in particular to a method for detecting the presence or level of one or more target nucleic acids in a sample, and also relates to a probe set and a kit containing one or more the probe sets.

### BACKGROUND

In recent years, the results of single cell research have revealed to us that the gene expression level in a single cell is greatly different from the average expression level of its surrounding cell population, that is, there is expression heterogeneity of cell populations in tissues. The expression level of RNA and its localization in tissues are often closely related to the regulation of growth and development of cells and tissues. Therefore, in order to better understand the function of genes and their regulatory networks while retaining spatial location information, quantitative detection of RNA is of great significance.

In the past, traditional in-situ hybridization could achieve in-situ detection of RNA. However, because the reaction products of this method are small molecules or precipitates, they can easily diffuse in the environment or even break away from the detection probe. Therefore, it is difficult to accurately locate the specific location of target molecule. At the same time, traditional methods have not been widely used in scientific research and diagnostic laboratories due to the complicated process of preparing probes and lack of sufficient sensitivity and specificity.

The subsequent development of single-molecule fluorescence in-situ hybridization has solved the above-mentioned problems of in-situ hybridization to a certain extent. The method is mainly based on the idea of detection that a plurality of detection probes labeled with multiple fluorophores are hybridized to a single strand of RNA, or a large number of detection probes labeled with a single fluorophore are hybridized to a single strand of RNA. Although this method can use the linear accumulation of fluorophores to achieve a certain intensity detection threshold, it is limited by the spectral overlap between different fluorophores that can be used for labeling, and the number of RNAs that can be detected simultaneously by this method is very limited. At the same time, the multiplex detection based on this method in tissues is usually interfered by background fluorescence and light scattering, which also limits the present application of this method in highly multiplexed detection of RNA.

The development of single-cell sequencing technology has further promoted this demand. However, it can only provide information about the heterogeneity of cell populations, but lacks accurate judgment of the exact cell type source and location information of RNA. Therefore, there is an urgent need for a technical means that can not only quantitatively analyze RNA expression abundance but also achieve in-situ detection.

### Contents of the Invention

The present application achieves specific detection of the presence or level of a target nucleic acid through uniquely designed V-type probe/C-type probe and padlock-type probe. The detection method or probe set of the present application can be combined with fluorescence microscopy or flow cytometry to simultaneously analyze multiple (e.g., 1, 5, 10, 15, 20, 50, 100 or more) target nucleic acids in a large number of cells.

Therefore, in a first aspect, the present application provides a method of detecting the presence or levels of one or more target nucleic acids in a sample, the method comprising:
(a) providing a detection sample suspected of containing one or more target nucleic acids, and, for each target nucleic acid, providing at least one (e.g., at least 2, at least 3, at least 5, or more) probe set, wherein the probe set comprises a first probe, a second probe, a padlock probe and a detection probe;
wherein, the first probe has a sequence comprising: (i) a first complementary sequence that specifically binds to the padlock probe; (ii) a first target-binding sequence that specifically binds to the target nucleic acid; (iii) optionally, a first linker sequence for linking the first complementary sequence and the first target-binding sequence;
the second probe has a sequence comprising in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence;
the padlock probe is a single-stranded nucleic acid, which comprises: (i) a backbone sequence, and (ii) a detection probe sequence; under a condition that allows hybridization or annealing, the padlock probe is capable of hybridizing with or annealing to the first complementary sequence of the first probe and the second complementary sequence of the second probe to form a circular polynucleotide with a nick;
the detection probe comprises a detectable label and the detection probe sequence or fragment thereof;
(b) contacting the detection sample with the first probe, the second probe, the padlock probe, and a ligase under a condition that allows the ligase to ligate a nucleic acid nick;
(c) performing rolling circle amplification of the product of step (b) by using an amplification enzyme under a condition that allows the amplification;
(d) contacting the product of the previous step with the detection probe under a condition that allows hybridization or annealing, and detecting a signal from the detection probe bound to the product;
(e) detecting the presence or level of the target nucleic acid in the detection sample based on the presence or level of the signal from the detection probe.

In certain embodiments, the first linker sequence does not bind to the target nucleic acid or padlock probe. In certain embodiments, the first target-binding sequence is located upstream or downstream of the first complementary sequence.

In certain embodiments, the second linker sequence does not bind to the target nucleic acid or padlock probe.

In certain embodiments, the first target-binding sequence is located upstream of the first complementary sequence. In certain preferred embodiments, the first probe comprises a first target-binding sequence, a first linker sequence and a first complementary sequence in the 5′ to 3′ direction. In certain preferred embodiments, the second probe comprises a second target-binding sequence, a second linker sequence and a second complementary sequence in the 5′ to 3′ direction. In such embodiments, the first probe and the second probe form a structure similar to “double-C” and are therefore referred to as “C-type probes” in certain embodiments herein.

In certain embodiments, the first target-binding sequence is located downstream of the first complementary sequence. In certain preferred embodiments, the first probe comprises a first complementary sequence, a first linker sequence and a first target-binding sequence in the 5′ to 3′ direction. In certain preferred embodiments, the second probe comprises a second target-binding sequence, a second linker sequence and a second complementary sequence in the 5′ to 3′ direction. In such embodiments, the first probe and the second probe form a structure similar to “v” and are therefore referred to as “V-type probes” in certain embodiments herein.

In certain embodiments, in step (b), the detection sample is allowed to contact with the first probe, the second probe, and the padlock probe. If the detection sample contains at least one target nucleic acid to be detected, the first target-binding sequence of the first probe and the second target-binding sequence of the second probe in the probe set are hybridized or annealed to the target nucleic acid, respectively. Subsequently, the padlock probe is hybridized or annealed to the first complementary sequence of the first probe and the second complementary sequence of the second probe, and forms a circular polynucleotide with a nick. Under a condition that allows ligase to connect a nucleic acid nick, the padlock probe forms a circular DNA by using the first complementary sequence of the first probe or the second complementary sequence of the second probe as a template.

In some embodiments, the padlock probe uses the first complementary sequence of the first probe as a template, and in this case, the backbone sequence of the padlock probe is located upstream of the detection probe sequence. In some embodiments, the padlock probe uses the second complementary sequence of the second probe as a template, and in this case, the detection probe sequence of the padlock probe is located upstream of the backbone sequence.

In such embodiments, the padlock probe is a single polynucleotide strand, and the padlock probe forms a circular polynucleotide with a nick after hybridizing or annealing to the first probe and the second probe. Compared with multiple (e.g., 2, 3, 4, 5, or more) polynucleotide strands, the single polynucleotide strand can better specifically bind to the target nucleic acid and reduce the probability of binding to non-specific nucleic acids, thereby reducing the background detection and obtaining a higher signal-to-noise ratio in the detection results.

In some embodiments, in step (c), by using the circular DNA as a template and the second complementary sequence as an amplification primer, and a nucleic acid polymerase is used to perform the rolling circle amplification of step (b) product under a condition that allows the amplification to obtain a rolling circle amplification product, and, the rolling circle amplification product contains at least one (e.g., 2, 3, 4, 5, 10, 15, 20 or more) sequences complementary to the detection probe sequence.

In certain embodiments, in step (d), the product of the previous step is contacted with the detection probe under a condition that allows hybridization or annealing, and the signal of the detection probe bound to the product is detected.

In certain embodiments, in step (e), the presence or level of the target nucleic acid in the detection sample is detected based on the presence or level of signal from the detection probe. The presence or level of signal from the detection probe can be determined by a variety of ways, such as by nano-SIM, flow/mass cytometry, fluorescence microscopy, etc.

In certain embodiments, the first complementary sequence of the first probe hybridizes to a first region of the padlock probe, and the second complementary sequence of the second probe hybridizes to a second region of the padlock probe, and there is a spacer sequence between the first region and the second region.

In certain embodiments, the spacer sequence has a length of 0 to 30 nt (e.g., 0 to 5 nt, 5 to 10 nt, 10 to 15 nt, 15 to 20 nt, 20 to 25 nt, 25 to 30 nt).

In certain embodiments, the spacer sequence has a length of 0 to 10 nt (e.g., 0 nt, 3 nt, 5 nt, 8 nt, 10 nt).

In certain embodiments, the first target-binding sequence and the second target-binding sequence are separated by 0 to 30 nt (e.g., 0 to 5 nt, 5 to 10 nt, 10 to 15 nt, 15 to 20 nt, 20 to 25 nt, 25 to 30 nt) on the target nucleic acid.

In certain embodiments, the first target-binding sequence and the second target-binding sequence are separated by 0 to 10 nt (e.g., 0 nt, 3 nt, 5 nt, 8 nt, 10 nt) on the target nucleic acid.

In certain embodiments, the detection sample is selected from the group consisting of a single cell, a cell population, a tissue, an organ, or any combination thereof. In certain embodiments, the detection sample is subjected to pretreatment. In certain embodiments, the pretreatment is selected from the group consisting of cell permeabilization, nucleic acid extraction, nucleic acid purification, and nucleic acid enrichment.

In certain embodiments, the target nucleic acid is DNA and/or RNA. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA, LncRNA (long non-coding RNA)).

In certain embodiments, the target nucleic acid is in a cell. In certain embodiments, the cell is selected from the group consisting of a eukaryotic cell (e.g., an animal cell, a plant cell, a fungal cell), a prokaryotic cell, an archaebacterial cell, an artificial cell, or any combination thereof.

In certain embodiments, the cell is a mammalian cell (e.g., a human cell). In certain preferred embodiments, the cell is subjected to pretreatment. In certain embodiments, the pretreatment is selected from the group consisting of cell permeabilization, nucleic acid extraction, nucleic acid purification, and nucleic acid enrichment.

In certain embodiments, the detectable label is selected from the group consisting of a fluorescent label, a bioluminescent label, a chemiluminescent label, an isotopic label, or any combination thereof.

In certain embodiments, the fluorescent label is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705).

In certain embodiments, the amplification enzyme is a nucleic acid polymerase (especially a template-dependent nucleic acid polymerase). In certain embodiments, the nucleic acid polymerase is a DNA polymerase, such as a thermostable DNA polymerase. In certain embodiments, the thermostable DNA polymerase is obtained from Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus and Aquifex aeolieus. In certain embodiments, the DNA polymerase is Φ29 polymerase.

In certain embodiments, the detection sample suspected of containing one or more target nucleic acids, the first probe, the second probe, the padlock probe, and the ligase are provided, and the detection sample is allowed to contact with the first probe, the second probe, the padlock probe and the ligase, and then the detection probe is provided; alternatively, the detection sample suspected of containing one or more target nucleic acids, the first probe, the second probe, and the padlock probe; the ligase and the detection probe are provided, and the detection sample is allowed to contact with them.

In certain embodiments, the ligase is selected from the group consisting of T4 DNA ligase, DNA ligase I, DNA ligase III and DNA ligase IV.

In certain embodiments, the ligase is T4 DNA ligase.

In certain embodiments, the first probe and the second probe each independently comprise or consist of naturally occurring nucleotides (e.g., deoxyribonucleotides or ribonucleotides), modified nucleotides, non-natural nucleotides (e.g., peptide nucleic acids (PNA) or locked nucleic acids), or any combination thereof.

In certain embodiments, the first probe and the second probe each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt.

In certain embodiments, the first complementary sequence and the second complementary sequence each independently have a length of 10 to 15 nt, 15 to 20 nt, 20 to 30 nt, 30 to 40 nt, 40 to 50 nt. In certain embodiments, the first complementary sequence and the second complementary sequence each independently have a length of 10 to 20 nt.

In certain embodiments, the first complementary sequence has a first portion complementary to the backbone sequence and a second portion complementary to the detection probe sequence.

In certain embodiments, the second complementary sequence has a third portion complementary to the backbone sequence and a fourth portion complementary to the detection probe sequence.

In certain embodiments, the first portion, the second portion, the third portion and the fourth portion each independently have a length of 0 nt to 15 nt. In certain embodiments, the first portion, the second portion, the third portion and the fourth portion each independently have a length of 5 nt to 10 nt (e.g., 5 nt, 6 nt, 7 nt, 8 nt, 9 nt, 10 nt).

In certain embodiments, the first and second linker sequences each independently have a length of 5 to 10 nt, 10 to 15 nt, 15 to 20 nt, 20 to 30 nt, 30 to 40 nt, 40 to 50 nt. In certain embodiments, the first and second linker sequences each independently have a length of 5 to 15 nt (e.g., 5 nt, 6 nt, 7 nt, 8 nt, 9 nt, 10 nt, 11 nt, 12 nt, 13 nt, 14 nt, 15 nt).

In certain embodiments, the first and second target-binding sequences each independently have a length of 12 to 15 nt, 15 to 20 nt, 20 to 30 nt, 30 to 40 nt, 40 to 50 nt. In certain embodiments, the first and second target-binding sequences each independently have a length of 12 to 30 nt.

In certain embodiments, the detection probes each independently comprise or consist of naturally occurring nucleotides (e.g., deoxyribonucleotides or ribonucleotides), modified nucleotides, non-natural nucleotides (e.g., peptide nucleic acids (PNA) or locked nucleic acids), or any combination thereof.

In certain embodiments, the detection probes each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt.

In certain embodiments, the detection probes each independently have a 3′-OH terminus; alternatively, the 3′-terminus of the probe is blocked; for example, the 3′-terminus of the detection probe is blocked by adding a chemical moiety (e.g., biotin or alkyl) to the 3′-OH of the last nucleotide of the probe, or by removing the 3′-OH of the last nucleotide of the probe, or by replacing the last nucleotide with a dideoxynucleotide.

In certain embodiments, the detection probes are each independently linear or have a hairpin structure.

In certain embodiments, the detection probes each independently bear a detectable label. In certain embodiments, the detection probes in the different probe sets bear different detectable labels.

In certain embodiments, the detection probe cannot be degraded by a nucleic acid polymerase (e.g., DNA polymerase).

In certain embodiments, the padlock probe is a linear continuous polynucleotide in its natural state.

In certain embodiments, the padlock probe is a circular polynucleotide with a nick when hybridized or annealed to the first probe and the second probe.

In certain embodiments, the padlock probes each independently comprise or consist of naturally occurring nucleotides (e.g., deoxyribonucleotides or ribonucleotides), modified nucleotides, non-natural nucleotides (e.g., peptide nucleic acids (PNA) or locked nucleic acids), or any combination thereof.

In certain embodiments, the padlock probes each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt.

In certain embodiments, the padlock probe cannot be degraded by a nucleic acid polymerase (e.g., DNA polymerase).

In a second aspect, the present application provides a probe set, which comprises a first probe, a second probe, a padlock probe and a detection probe;
wherein, the first probe has a sequence comprising: (i) a first complementary sequence that specifically binds to the padlock probe; (ii) a first target-binding sequence that specifically binds to a target nucleic acid; (iii) optionally, a first linker sequence for linking the first complementary sequence and the first target-binding sequence;
the second probe has a sequence comprising in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence;
the padlock probe is a single-stranded nucleic acid, which comprises: (i) a backbone sequence, and (ii) a detection probe sequence; under a condition that allows hybridization or annealing, the padlock probe is capable of hybridizing or annealing to the first complementary sequence of the first probe and the second complementary sequence of the second probe to form a circular polynucleotide with a nick;
the detection probe comprises a detectable label and the detection probe sequence or fragment thereof.

In certain embodiments, the first linker sequence does not bind to the target nucleic acid or the padlock probe. In certain embodiments, the first target-binding sequence is located upstream or downstream of the first complementary sequence.

In certain embodiments, the second linker sequence does not bind to the target nucleic acid or the padlock probe.

In certain embodiments, the first target-binding sequence is located upstream of the first complementary sequence. In certain preferred embodiments, the first probe comprises the first target-binding sequence, the first linker sequence and the first complementary sequence in the 5′ to 3′ direction. In certain preferred embodiments, the second probe comprises the second target-binding sequence, the second linker sequence and the second complementary sequence in the 5′ to 3′ direction. In such embodiments, the first probe and the second probe form a structure similar to “double-C” and are therefore referred to as “C-type probes” in certain embodiments herein.

In certain embodiments, the first target-binding sequence is located downstream of the first complementary sequence. In certain preferred embodiments, the first probe comprises the first complementary sequence, the first linker sequence and the first target-binding sequence in the 5′ to 3′ direction. In certain preferred embodiments, the second probe comprises the second target-binding sequence, the second linker sequence and the second complementary sequence in the 5′ to 3′ direction. The first probe and the second probe form a structure similar to “v” and are therefore referred to as “V-type probes” in certain embodiments herein.

In a third aspect, the present application provides a kit, which comprises one or more probe sets as described above.

In certain embodiments, the kit further comprises a ligase, an amplification enzyme, a reagent for nucleic acid amplification, a reagent for rolling circle amplification, a reagent for detecting a fluorescent signal, or any combination thereof.

In certain embodiments, the ligase is selected from the group consisting of T4 DNA ligase, DNA ligase I, DNA ligase III and DNA ligase IV. In certain embodiments, the ligase is T4 DNA ligase.

In certain embodiments, the amplification enzyme is a nucleic acid polymerase (especially a template-dependent nucleic acid polymerase). In certain embodiments, the nucleic acid polymerase is a DNA polymerase, such as a thermostable DNA polymerase. In certain embodiments, the thermostable DNA polymerase is obtained from: Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus and Aquifex aeolieus. In certain embodiments, the DNA polymerase is Φ29 polymerase.

In certain embodiments, the detection probe has characteristics as described above.

In certain embodiments, the reagent for nucleic acid amplification comprises working buffer of enzyme (e.g., nucleic acid polymerase), dNTPs (labeled or unlabeled), water, solution containing ion (e.g., Mg²⁺), single-stranded DNA binding protein, or any combination thereof.

In certain embodiments, the reagent for rolling circle amplification is selected from RNase-free water, dNTPs (labeled or unlabeled), RNase inhibitor, or any combination thereof.

In certain embodiments, the kit is used to detect the presence or levels of one or more target nucleic acids in a sample.

### Definition of Terms

In the present invention, unless otherwise stated, scientific and technical terms used herein have the meanings commonly understood by those skilled in the art. Moreover, the procedures used herein, such as those of molecular genetics, nucleic acid chemistry, chemistry, molecular biology, biochemistry, cell culture, microbiology, cell biology, genomics and recombinant DNA are routine procedures widely used in the corresponding fields. Meanwhile, in order to better understand the present invention, definitions and explanations of relevant terms are provided below.

As used herein, the term “target nucleic acid” is any polynucleotide molecule (e.g., DNA molecule; RNA molecule, modified nucleic acid molecule, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA, LncRNA (long non-coding RNA), etc.). In some embodiments, the target nucleic acid is a splice variant of RNA molecule (e.g., mRNA, pre-mRNA, etc.). Therefore, a suitable target nucleic acid may be a unspliced RNA (e.g., pre-mRNA, mRNA), a partially spliced RNA or a fully spliced RNA, etc.

As used herein, the term “target-binding sequence” refers to a sequence on the first or second probe that is complementary to a target nucleic acid. Typically, the first target-binding sequence and the second target-binding sequence are complementary to adjacent positions on the target nucleic acid, for example, there is typically a distance of no more than 10 nt (e.g., no more than 9, 8, 7, 6, 5, 4, 3, 2 or 1 nt). The first target-binding sequence and the second target-binding sequence may also be in adjacent positions (i.e., the distance is 0 nt). The length of each target-binding sequence is usually about 12 to 30 nt, such as 15 to 25 nt, 18 to 23 nt, 18 to 21 nt.

As used herein, the terms “first probe” and “second probe” have a sequence comprising: (i) a complementary sequence that specifically binds to the padlock probe; (ii) a target-binding sequence that specifically binds to the target nucleic acid; (ii) optionally, a linker sequence for linking the complementary sequence and the target-binding sequence. In some embodiments, the linker sequence is a poly-A sequence. In some embodiments, the linker sequence has a length of 5 to 20 nt, for example, 8 to 15 nt, 10 to 12 nt.

As used herein, the term “padlock probe” is a continuous single-stranded nucleic acid, which comprises: (i) a backbone sequence, and (ii) a detection probe sequence. Under a condition that allows hybridization or annealing, the padlock probe can hybridize or anneal to the first probe and the second probe to form a circular polynucleotide with a nick. In some embodiments, the backbone sequence is located upstream of the detection probe sequence, and in such case, the nick is located in a first region complementary to the first probe. In some embodiments, the backbone sequence is located downstream of the detection probe sequence, and in such case, the nick is located in a second region complementary to the second probe.

As used herein, the term “rolling circle amplification” refers to the enzymatic synthesis of dNTPs into single-stranded DNA using circular DNA as a template through a primer (complementary to part of the circular template), in which the single-stranded DNA comprises many repetitive template complementary fragments. This technology is known in the art (see, for example, Baner et al, Nucleic Acids Research, 26:5073-5078, 1998; Lizardi et al, Nature Genetics 19:226, 1998; Schweitzer et al. Proc. Natl Acad. Sci. USA 97:10113-119, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801).

As used herein, the term “detection probe” determines the presence and level of a nucleic acid to be tested in a cell by direct or indirect contact with the nucleic acid to be tested under a condition of hybridization or annealing. The detection probe comprises a detectable label that can be measured and quantified. In some embodiments, the detection probe bears a single detectable label. In some embodiments, the detection probe bears a plurality of (e.g., 3) detectable labels, in which the first detectable label is capable of specifically binding to a first nucleic acid to be tested, and the remaining detectable labels specifically bind or do not bind to other nucleic acids to be tested.

As used herein, the term “detectable label” refers to any component capable of providing a detection signal under a detection condition, and comprises directly and indirectly detectable labels. Detectable labels useful in the methods described herein comprise any component that can be detected indirectly or directly by spectroscopic, photochemical, biochemical, immunochemical, electrical, optical, chemical or other means. Examples thereof comprise: antigen label (e.g., digoxigenin (DIG), fluorescein, dinitrophenol (DNP), etc.), biotin capable of being stained with labeled streptavidin conjugate, fluorescent dye (e.g., fluorescein, Texaco red, rhodamine, fluorophore label such as Alex Fluor label, etc.), radioactive label (e.g., ¹²⁵i, ³⁵S, ¹⁴C, or ³²P), enzyme (e.g., peroxidase, alkaline phosphatase, galactase and other enzymes commonly used in ELISA), fluorescent protein (e.g., green fluorescent protein, red fluorescent protein, yellow fluorescent protein, etc.), synthetic polymer capable of chelating metal, colorimetric label, etc.

Wherein, the detectable label can generate a signal detected by a photodetector (e.g., flow cytometer, fluorescence microscope). Flow or mass cytometry can be used to quantify parameters when it is used to determine the presence of cell surface protein or conformational or post-translational modification thereof, or intracellular or secreted protein. When it is used in single-cell multi-parameter and multi-cell multi-parameter multiplex assays, the type of input cells can be identified and parameters can be read by quantitative imaging and fluorescence confocal microscopy, and the methods of fluorescence confocal microscopy are known in the art, see, for example, Confocal Microscopy Methods and Protocols (Methods in Molecular Biology, Volume 122, Humana Press, 1998).

As used herein, “cell” refers to any type of cell from prokaryotes (e.g., bacteria, fungi, protozoa), eukaryotes (e.g., plants, animals), or palaeobios, including cells from tissues, organs, and living tissues, recombinant cells, cells from cell lines cultured in vitro, and cellular fragments containing nucleic acids, cellular components. The cells also include artificial cells, such as nucleic acid-encapsulating particles, liposomes, polymers or microcapsules. The cells may include fixed cells, permeabilized cells or viable cells. The methods described herein can be performed on samples containing, for example, single cells, cell populations, or tissues or organs. The “viable cells” as used herein refers to naturally occurring or modified intact cells. The viable cells can be isolated from other cells, mixed with other cells in culture or in tissues (partial or complete) or organisms.

As used herein, the terms “polynucleotide,” “oligonucleotide,” “nucleic acid,” and “nucleic acid molecule” comprise polymeric forms of nucleotides (e.g., ribonucleotides, deoxyribonucleotides) of any length, and these terms can be used interchangeably. The terms comprise triple-stranded, double-stranded, and single-stranded DNA, as well as triple-stranded, double-stranded, and single-stranded RNA. The terms also comprise modified (e.g., methylated and/or capped) or unmodified forms.

As used herein, the terms “hybridization” and “complementarity” refer to the formation of a complex between nucleotide sequences, and the complex can be formed by Watson-Crick base pairing. Those skilled in the art will understand that a sequence capable of hybridizing or being complementary to a target nucleic acid does not need to be completely complementary to its target nucleic acid sequence. In many cases, a stable hybrid can be formed even if there are approximately 10% mismatched bases in the hybrid. Thus, a sequence that “hybridizes” and is “complementary” to a target nucleic acid sequence has 90% or greater homology to a sequence that is completely complementary to its target nucleic acid sequence.

As used herein, the term “CEU” refers to enzyme activity unit. CEU (cohesive end ligation unit) can refer to the amount of enzyme required to ligate 50% of HindIII-digested nucleic acid fragments within 30 minutes at 16° C. Generally, the lower the amount of enzyme used, the higher the enzyme activity demonstrated. Enzyme activity units also include U (U-activity unit), etc.

### Beneficial Effects of the Invention

The probe set and detection method of the present application are capable of simultaneously detecting the presence or levels of a plurality of target nucleic acids. Compared with the prior art, it has one or more beneficial effects selected from the following: (1) reducing the number of probes used for detection; (2) shortening the detection time; (3) reducing the amount of enzyme (e.g., ligase); (4) improving hybridization efficiency; (5) being capable of detecting shorter target nucleic acids (e.g., the target nucleic acid to be detected may have a length as low as 12 nt/bp); (6) improving detection accuracy, i.e., reducing non-specific detection results.

The embodiments of the present invention will be described in detail below with reference to the accompanying drawings and examples, but those skilled in the art will understand that the following drawings and examples are only used to illustrate the present invention and do not limit the scope of the present invention. Various objects and advantageous aspects of the present invention will become apparent to those skilled in the art from the accompanying drawings and the following detailed description of preferred embodiments.

### BRIEF DESCRIPTION OF THE DRAWINGS

FIG. 1 shows the in-situ detection results of ALB RNA on different cells by the V-type probe set, in which FIG. 1A shows the detection results of HepG2 cells, and FIG. 1B shows the detection results of SKBR3 cells.

FIG. 2 shows the in-situ detection results of HER2 RNA, UBC RNA and dapB RNA on the SKBR3 cell line by the V-type probe set, in which dapB is the negative control gene, indigo fluorescence is the detection result of dapB; red fluorescence is the detection results of HER2; and the silver fluorescence is the detection result of UBC.

FIG. 3 shows the results of in-situ detection of HER2 RNA on the SKBR3 cell line by the V-type probe set, in which FIG. 3A shows the detection results of the V-type probe and the RNA sequence at hybridization length of 10 nt, FIG. 3B shows the detection results of the V-type probe and the RNA sequence at hybridization length of 12 nt, and FIG. 3C shows the detection results of the V-type probe and the RNA sequence at hybridization length of 15 nt, and FIG. 3D shows the detection results of the V-type probe and the RNA sequence at hybridization length of 20 nt.

FIG. 4 shows the in-situ detection results of HER2 RNA on SKBR3 cell line by the V-type probe set, in which FIG. 4A shows the detection results when the spacer sequence has is of 0 nt in length, FIG. 4B shows the detection result when the spacer sequence is of 5 nt in length, and FIG. 4C shows the detection result when the spacer sequence is of 10 nt in length.

FIG. 5 shows the in-situ detection results of HER2 RNA on the SKBR3 cell line by the V-type probe set, in which the V-type probe 2 has hybridization of 9 bp with the backbone sequence of the padlock probe, and hybridization of 8 bp with the detection probe sequence of the padlock probe, wherein FIG. 5A shows that the V-type probe 1 has hybridization of 8 bp with the backbone sequence of the padlock probe, and hybridization of 7 bp with the detection probe of the padlock probe; FIG. 5B shows that the V-type probe 1 has hybridization of 7 bp with the backbone sequence of the padlock probe, and hybridization of 6 bp with the detection probe of the padlock probe; and FIG. 5C shows that the V-type probe 1 has hybridization of 6 bp with the backbone sequence of the padlock probe, and hybridization of 6 bp with the detection probe of the padlock probe.

FIG. 6 shows the results of in-situ detection of HER2 RNA on the SKBR3 cell line by the V-type probe set, in which the V-type probe 1 has hybridization of 7 bp with the backbone sequence of the padlock probe, and hybridization of 6 bp with the detection probe sequence of the padlock probe, wherein FIG. 6A shows that the V-type probe 2 has hybridization of 9 bp with the backbone sequence of the padlock probe, and hybridization of 8 bp with the detection probe of the padlock probe; FIG. 6B shows that the V-type probe 2 has hybridization of 8 bp with the backbone sequence of the padlock probe, and hybridization of 7 bp with the detection probe of the padlock probe; FIG. 6C shows that the V-type probe 2 has hybridization of 7 bp with the backbone sequence of the padlock probe, and hybridization of 6 bp with the detection probe of the padlock probe; FIG. 6D shows that the V-type probe 2 has hybridization of 6 bp with the backbone sequence of the padlock probe, and hybridization of 6 bp with the detection probe of the padlock probe.

FIG. 7 shows the results of in-situ detection of HER2 RNA on the SKBR3 cell line by the V-type probe set, in which the V-type probe 2 has hybridization of 8 bp with the backbone sequence of the padlock probe, and hybridization of 7 bp with the detection probe sequence of the padlock probe, wherein FIG. 7A shows that the V-type probe 1 has hybridization of 7 bp with the backbone sequence of the padlock probe, and hybridization of 8 bp with the detection probe of the padlock probe; FIG. 7B shows that the V-type probe 1 has hybridization of 7 bp with the backbone sequence of the padlock probe, and hybridization of 7 bp with the detection probe of the padlock probe; FIG. 7C shows that the V-type probe 1 has hybridization of 6 bp with the backbone sequence of the padlock probe, and hybridization of 7 bp with the detection probe of the padlock probe.

FIG. 8 shows the results of in-situ detection of HER2 RNA on the SKBR3 cell line by the C-type probe set.

FIG. 9 shows the schematic diagram of in-situ detection of RNA by the V-type probe of the present application.

Wherein, the sequence of V-type probe 1 comprises in the 5′ to 3′ direction: (i) a first complementary sequence that specifically binds to the padlock probe; (ii) a first target-binding sequence that specifically binds to the target nucleic acid; (iii) optionally, a first linker sequence for linking the first complementary sequence and the first target-binding sequence.

The sequence of V-type probe 2 comprises in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence.

The padlock probe is a single-stranded nucleic acid, which comprises: (i) a backbone sequence, and (ii) a detection probe sequence; under a condition that allows hybridization or annealing, the padlock probe is capable of hybridizing or annealing to a first complementary sequence of V-type probe 1 and a second complementary sequence of V-type probe 2 to form a circular polynucleotide with a nick.

The detection probe comprises a detectable label and the detection probe sequence or fragment thereof.

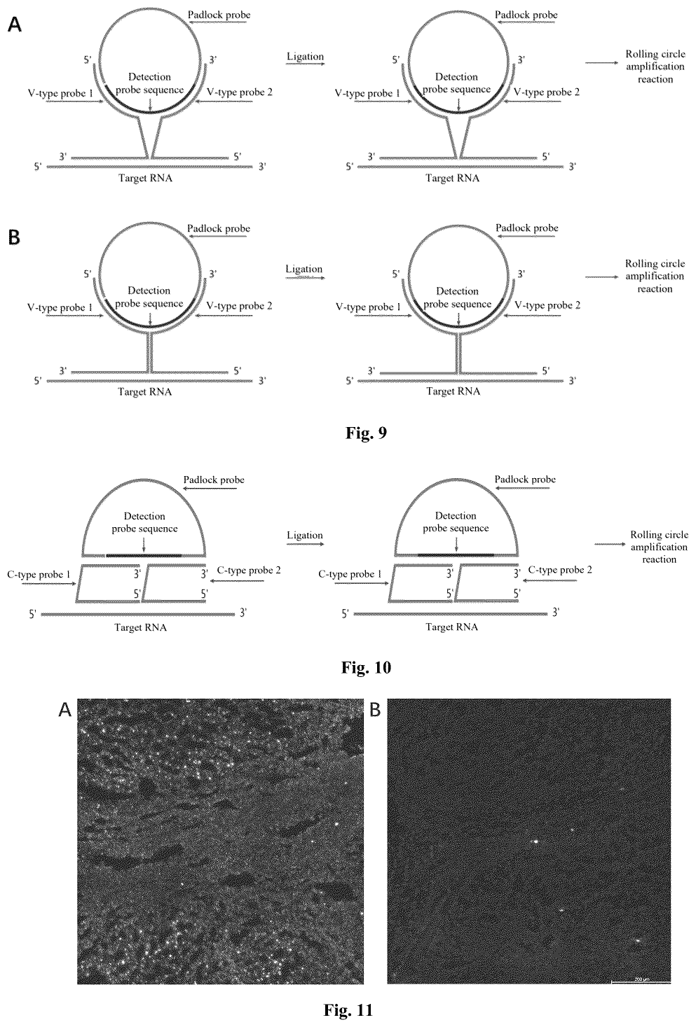

Under a condition that allows hybridization or annealing, the V-type probe 1 and the V-type probe 2 hybridize to the target RNA through the first target-binding sequence and the second target-binding sequence, respectively. Subsequently, a first region and a second region of the padlock probe hybridize with the first complementary sequence of V-type probe 1 and the second complementary sequence of V-type probe 2, respectively, and there is a spacer sequence between the first region and the second region; and, the padlock probe changes from a linear single-stranded nucleic acid to a circular single-stranded nucleic acid with a nick. Under a condition that allow ligase to ligate a nucleic acid nick, the padlock probe forms a circular DNA by using the first complementary sequence of V-type probe 1 or the second complementary sequence of V-type probe 2 as a template. Under a condition that allows amplification, rolling circle amplification is performed using the circular DNA as a template and the second complementary sequence as an amplification primer to obtain a rolling circle amplification product, and the rolling circle amplification product comprises a sequence complementary to the detection probe sequence. Under a condition that allows hybridization or annealing, a detection probe is added and a signal from the detection probe bound to the product is detected. Based on the presence or level of the signal from the detection probe, the presence or level of the target nucleic acid in the detection sample is detected. Wherein, FIG. 9A shows that the spacer sequence has a length greater than 0 nt; FIG. 9B shows that the spacer sequence has a length equal to 0 nt.

FIG. 10 shows the schematic diagram of in-situ detection of RNA by the C-type probe of the present application.

Wherein, the sequence of C-type probe 1 comprises in the 5′ to 3′ direction: (i) a first target-binding sequence that specifically binds to the target nucleic acid; (ii) a first complementary sequence that specifically binds to the padlock probe; (iii) optionally, a first linker sequence for linking the first complementary sequence and the first target-binding sequence.

The sequence of C-type probe 2 comprises in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence.

The padlock probe is a single-stranded nucleic acid, which comprises: (i) a backbone sequence, and (ii) a detection probe sequence; under a condition that allows hybridization or annealing, the padlock probe is capable of hybridizing and annealing to the first complementary sequence of C-type probe 1 and the second complementary sequence of C-type probe 2 to form a circular polynucleotide with a nick.

The detection probe comprises a detectable label and the detection probe sequence or fragment thereof.

Under a condition that allows hybridization or annealing, C-type probe 1 and C-type probe 2 hybridize to the target RNA through the first target-binding sequence and the second target-binding sequence, respectively. Subsequently, the first region and the second region of the padlock probe hybridize with the first complementary sequence of the C-type probe 1 and the second complementary sequence of the C-type probe 2, respectively, and there is a spacer sequence between the first region and the second region; and, the padlock probe changes from a linear single-stranded nucleic acid to a circular single-stranded nucleic acid with a nick. Under a condition that allows ligase to ligate a nucleic acid nick, the padlock probe forms a circular DNA by using the first complementary sequence of C-type probe 1 or the second complementary sequence of C-type probe 2 as a template. Under a condition that allows amplification, rolling circle amplification is performed using the circular DNA as a template and the second complementary sequence as an amplification primer to obtain a rolling circle amplification product, and the rolling circle amplification product comprises a sequence complementary to the detection probe sequence. Under a condition that allows hybridization or annealing, a detection probe is added and a signal from the detection probe bound to the product is detected. Based on the presence or level of the signal from the detection probe, the presence or level of the target nucleic acid in the detection sample is determined.

FIG. 11 shows the results of in-situ detection of UBC RNA on human liver tissue by the V-type probe set, in which FIG. 11A shows the detection results with the addition of the padlock probe, and FIG. 11B shows the detection results without the addition of the padlock probe.

### SPECIFIC MODELS FOR CARRYING OUT THE INVENTION

The present invention will be described by referring to the following Examples that are intended to illustrate the present invention (rather than limiting the present invention).

Unless otherwise indicated, the experiments and methods described in the examples were performed essentially according to conventional methods well known in the art and described in various references. For example, for conventional techniques such as immunology, biochemistry, chemistry, molecular biology, microbiology, cell biology, genomics and recombinant DNA used in the present invention, references may be seen in: Sambrook, Fritsch and Maniatis, MOLECULAR CLONING: A LABORATORY MANUAL, 2^{nd} edition (1989); CURRENT PROTOCOLS IN MOLECULAR BIOLOGY, edited by F. M. Ausubel et al., (1987); “METHODS IN ENZYMOLOGY” series, Academic Publishing Company: “PCR 2: A PRACTICAL APPROACH”, edited by M. J. MacPherson, B. D. Hames, and G. R. Taylor (1995), and ANIMAL CELL CULTURE, edited by R. I. Freshney (1987).

In addition, for those without giving the specific conditions in the examples, they were carried out according to conventional conditions or conditions recommended by manufacturers. For reagents or instruments used without giving manufactures, they were all conventional products that could be purchased commercially. Those skilled in the art would appreciate that the examples describe the present invention by way of example and are not intended to limit the scope of the present invention. All publications and other references mentioned herein are incorporated by reference in their entirety.

### Example 1: Comparison of In-Situ Detection Experiments of V-Type Probes

### 1.1 In-Situ Detection Experiments of V-Type Probes

In this example, designed V-type probes were used to conduct in-situ detection experiments of ALB (Albumin serum albumin) RNA on HepG2 cells (liver cancer cells) and SKBR3 cells (breast cancer cells). The detection principle of the probes was shown in FIG. 9.

First, a V-type probe stock solution was prepared by adding DEPC water, in which the specific probe sequences used were shown in Table 1. Cell slides were prepared from HepG2 cells and SKBR3 cells, respectively (preparation of cell slides: when the cell growth density reached 80% to 90%, the cells were digested to form a single cell suspension; sterilized slides were taken and placed in culture dish, complete culture medium was added to the dish, and then the cells were dropped onto the slide so that the cell suspension was evenly distributed on the glass slide, and then the cells were cultured in a CO₂ incubator for 12 to 48 hours; when the cell growth density reached 70% to 80%, the culture was stopped, and the cells were fixed with 4% paraformaldehyde solution; after the cell slides were prepared, they were stored in a −80° C. refrigerator, taken out before use), and the subsequent processing areas were defined using use an immunohistochemistry pen. HepG2 cells and SKBR3 cells were then treated, permeabilized with 0.5% Triton X-100 in 1×PBS for 10 min, and washed three times with DEPC-PBS-Tween (0.1%). Subsequently, the V-type probes 1 and 2 were hybridized with the target RNA. The reagents used were shown in Table 2. The hybridization was carried out for 1 hour in a 37° C. constant temperature incubator, then rinsing was performed three times with 1×Hyb buffer 2 (2×SSC, 20% formamide), and washing was performed three times with DEPC-PBS-Tween. Then the padlock probe (the padlock probe had been 5′-phosphorylated in advance by Shenggong Bioengineering (Shanghai) Co., Ltd.) was subjected to hybridization, in which the reagents used were shown in Table 3, the hybridization was carried out for 1 hour in a 37° C. constant temperature incubator, and washing was performed three times with DEPC-PBS-Tween. Then, the padlock probe was subjected to ligation, in which the reagents used were shown in Table 4, the hybridization was carried out in a 37° C. constant temperature incubator for 1 hour, and washing was performed three times with DEPC-PBS-Tween. Then, rolling circle amplification was performed, in which the reagents used were shown in Table 5, the hybridization was carried out in a 37° C. constant temperature incubator for 1 hour, and washing was performed three times with DEPC-PBS-Tween. The detection probe (the 5′ end of the detection probe had Cy3) was added, in which the reagents used were shown in Table 6, the incubation was carried out at room temperature for 30 minutes, and washing was performed three times with DEPC-PBS-Tween.

The cell slides passed through 70%, 85%, and 100% gradient alcohol solutions in sequence at room temperature in the dark, in which dehydration was carried out at each concentration for 2 minutes, and then naturally air-dried, and the slides were sealed with an antifluorescent quencher (SlowFade Gold Antifade Mountant, which was purchased from Invitrogen, Cat. No. S36936) containing DAPI (DAPI could penetrate the cell membrane to stain the nucleus, and could emit blue fluorescence under detection conditions). Microscopic imaging was performed using a fluorescence microscope.

TABLE 1
Probe sequences

| Description | Sequence | SEQ ID NO: |
|---|---|---|
| ALB detection | AGTAGCCGTGACTATCGACT | 1 |
| probe | | |
| ALB padlock | ATTAGCGGTCCGTCTAGGAGAGTAGTACAGCAGCCGTCAAGAGTGT | 2 |
| probe | AGTAGCCGTGACTATCGACT | |
| RNA site 1 | TGAGAGAAATGAATGCTTCTTGCAACACAAAGATGACAAC | 3 |
| V-type probe 1-1 | CGCTAATAGTCGATAAAAAAAAAAAAGAAGCATTCATTTCTCTCA | 4 |
| V-type probe 2-1 | GTTGTCATCTTTGTGTTGCAAAAAAAAAAATGGCTACTACACTCTT | 5 |
| RNA site 2 | TAGTGACAGATCTTACCAAAGTCCACACGGAATGCTGCCA | 6 |
| V-type probe 1-2 | CGCTAATAGTCGATAAAAAAAAAAATTTGGTAAGATCTGTCACTA | 7 |
| V-type probe 2-2 | TGGCAGCATTCCGTGTGGACAAAAAAAAAATGGCTACTACACTCTT | 8 |
| RNA site 3 | CAGATCCTCATGAATGCTATGCCAAAGTGTTCGATGAATT | 9 |
| V-type probe 1-3 | CGCTAATAGTCGATAAAAAAAAAAAATAGCATTCATGAGGATCTG | 10 |
| V-type probe 2-3 | AATTCATCGAACACTTTGGCAAAAAAAAAATGGCTACTACACTCTT | 11 |
| RNA site 4 | GAAGACTATCTATCCGTGGTCCTGAACCAGTTATGTGTGT | 12 |
| V-type probe 1-4 | CGCTAATAGTCGATAAAAAAAAAAAACCACGGATAGATAGTCTTC | 13 |
| V-type probe 2-4 | ACACACATAACTGGTTCAGGAAAAAAAAAATGGCTACTACACTCTT | 14 |
| RNA site 5 | GAGAGACAAATCAAGAAACAAACTGCACTTGTTGAGCTCG | 15 |
| V-type probe 1-5 | CGCTAATAGTCGATAAAAAAAAAAATGTTTCTTGATTTGTCTCTC | 16 |
| V-type probe 2-5 | CGAGCTCAACAAGTGCAGTTAAAAAAAAAATGGCTACTACACTCTT | 17 |

TABLE 2
Hybridization of V-type probes

| Hybridization of V-type probe | stock | | final | | 1 x | |
|---|---|---|---|---|---|---|
| DEPC H₂O | | | | | 21.25 | ul |
| 2x Hyb buffer1 (12x SSC, 20% | 2 | x | 1 | x | 25 | ul |
| formamide) | | | | | | |
| ALB V-type probe 1 (5targets) | 2 | uM | 0.05 | uM | 1.25 | ul |
| ALB V-type probe 2 (5targets) | 2 | uM | 0.1 | uM | 2.5 | ul |
| Total | | | | | 50 | ul |

TABLE 3
Hybridization of padlock probes

| Hybridization of padlock probe | stock | | final | | 1 x | |
|---|---|---|---|---|---|---|
| DEPC H₂O | | | | | 28.75 | ul |
| T4 DNA ligase buffer(Thermo) | 10 | x | 1 | x | 5 | ul |
| 2.5M NaCl in 2% Tween-20 | 10 | x | 1 | x | 5 | ul |
| ALB padlock probe | 2 | uM | 0.2 | uM | 5 | ul |
| BSA | 2 | ug/ul | 0.2 | ug/ul | 5 | ul |
| RiboLock RNase Inhibitor | 40 | U/ul | 1 | U/ul | 1.25 | ul |

| | | |
|---|---|---|
| Total | 50 | ul |

TABLE 4
Ligation of padlock probe

| Ligation | stock | | final | | 1 x | |
|---|---|---|---|---|---|---|
| DEPC H₂O | | | | | 27.5 | ul |
| T4 DNA ligase buffer(Thermo) | 10 | x | 1 | x | 5 | ul |
| 2.5M NaCl in 2% Tween-20 | 10 | x | 1 | x | 5 | ul |
| ATP | 10 | mM | 1 | mM | 5 | ul |
| BSA | 2 | ug/ul | 0.2 | ug/ul | 5 | ul |
| 0.5 U/ul T4 DNA | 0.5 | U/ul | 0.0125 | U/ul | 1.25 | ul |
| ligase(Thermo) in | | | | | | |
| 50% Glycerol | | | | | | |
| RiboLock RNase Inhibitor | 40 | U/ul | 1 | U/ul | 1.25 | ul |
| Total | | | | | 50 | ul |

TABLE 5
Rolling circle amplification

| Rolling circle amplification | stock | | final | | 1 x | |
|---|---|---|---|---|---|---|
| DEPC H₂O | | | | | 26.75 | ul |
| Φ29 buffer | 10 | x | 1 | x | 5 | ul |
| Glycerol | 50% | | 5% | | 5 | ul |
| dNTP | 25 | mM | 1 | mM | 2 | ul |
| BSA | 2 | ug/ul | 0.2 | ug/ul | 5 | ul |
| Φ29 polymerase (Thermo) | 10 | u/ul | 1 | u/ul | 5 | ul |
| RiboLock RNase Inhibitor | 40 | u/ul | 1 | u/ul | 1.25 | ul |
| Total | | | | | 50 | ul |

TABLE 6
Hybridization of detection probe

| Detection | stock | | final | | 1 x | |
|---|---|---|---|---|---|---|
| H₂O | | | | | 22.5 | ul |
| 2x Hyb buffer2 (4x SSC, 40% formamide) | 2 | x | 1 | x | 25 | ul |
| ALB detection probe | 2 | uM | 0.1 | uM | 2.5 | ul |

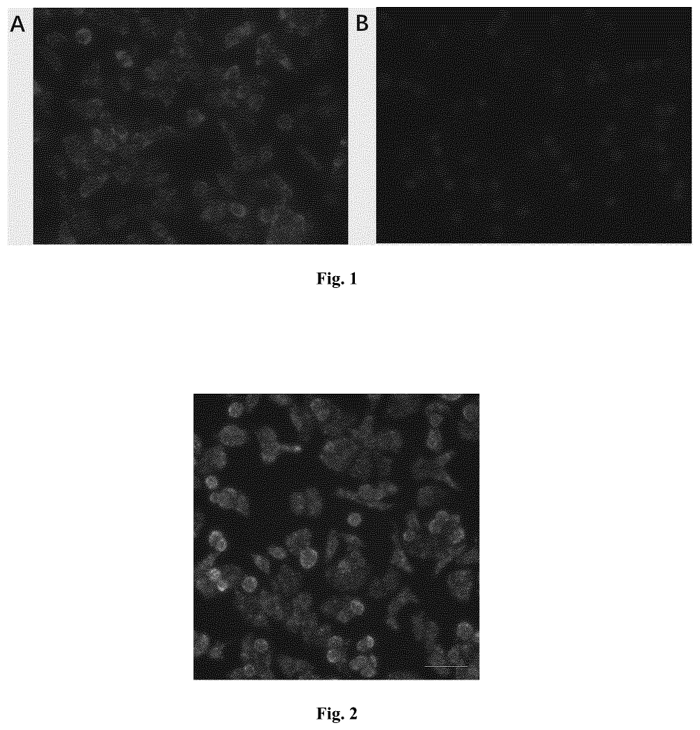

The results were shown in FIG. 1. ALB was clearly detected on HepG2 (FIG. 1A), and the detection amount was 71.47 fluorescence signal points per cell, while it was basically not detected on SKBR3 (FIG. 1B), and the amount was 0.01 fluorescent signal points per cell (considered as not detected).

ALB was highly expressed on HepG2 cells, but various databases and literature showed that it was not expressed on SKBR3 cells, and the data index NX values of ALB for RNA expression on HepG2 and SKBR3 as shown in the HPA database (https://www.proteinatlas.org/ENSG00000163631-ALB/scell) were listed here as examples, and the values were 315.9 and 0, respectively (when NX index was below 1.0, it was considered that the protein corresponding to the RNA was not expressed), which were in good agreement with the results obtained in this experiment using our probe system and experimental conditions. This not only showed that this probe system had good detection efficiency for highly expressed genes, but also had high specificity and would not produce false positive detection results for genes that were not expressed. Moreover, the expression level of gene could be detected.

1.2 In-Situ Detection Experiments of Probes with Other Design Methods

In this example, two independent sequences were separately synthesized, which were the detection probe sequence and the backbone sequence in the ALB padlock probe, and their specific sequences were shown in Table 7.

TABLE 7
Detection probe sequence and backbone sequence

| | | SEQ |
|---|---|---|
| Description | Sequence | ID NO: |
| ALB detection probe | AGTAGCCGTGACTATCGACT | 1 |
| sequence | | |
| ALB backbone | ATTAGCGGTCCGTCTAGGAGAGT | 18 |
| sequence | AGTACAGCAGCCGTCAAGAGTGT | |

The two sequences synthesized above were used together with the ALB padlock probe, V-type probe 1-1, and V-type probe 2-1 described above to perform in-situ detection experiments, in which the experimental steps and the reagents and raw materials used were the same as those described above. Wherein, 4 experiment groups were set respectively, in which Experimental group 1 used 2.5 CEU/ul ligase, and the three experimental steps were performed for a first set of incubation times; Experimental group 2 used 2.5 CEU/ul ligase, and the three experimental steps were performed for a second set of incubation times; Experimental group 3 used 10 CEU/ul ligase, and the three experimental steps were performed for a first set of incubation times; Experimental group 4 used 10 CEU/ul ligase, and three experimental steps were performed for a second set of incubation times. The specific incubation times were shown in Table 8.

TABLE 8
Incubation times for different steps

| | First set of | Second set of |
|---|---|---|
| Experimental steps | incubation times | incubation times |
| Hybridization of padlock probe | 2 h | 1 h |
| Ligation of padlock probe | 1 h | 1 h |
| Rolling circle amplification | 3 h | 1 h |
| Total time | 6 h | 3 h |

The experimental results showed that Experimental groups 1 to 4 all had a large amount of non-target detection, and compared with Experimental groups 1 and 2, Experimental groups 3 and 4 had fewer amount of target detection and more non-target detection. At the same time, Experimental group 1 and 2 showed no significant difference in detection efficiency.

Therefore, compared with the probes designed in other ways, the probe set designed in the present application improved the DNA ligase ligation efficiency and reduced the time required for the reaction and the amount of DNA ligase. On the other hand, the probe cost caused by two short nucleic acid sequences was reduced, and non-specific hybridization of short nucleic acid sequences could be avoided, thereby greatly improving the specificity of detection.

### Example 2: Triple Detection of V-Type Probes

In this example, V-type probes for the three genes HER2&UBC&dapB were designed, respectively, and multiplex in-situ detection experiments of the three genes HER2&UBC&dapB on the SKBR3 cell line was carried out. The experimental steps and reagents used were the same as those described in Example 1.1, and the specific probes used were shown in Table 9 below.

TABLE 9
Probe sequences

| Description | Sequence | SEQ ID NO: |
|---|---|---|
| HER2 RNA site 1 | CTCACCTACCTGCCCACCAATGCCAGCCTGTCCTTCCTGC | 19 |
| HER2 V-type probe | CGCTAATAGTCGATAAAAAAAAAAATTGGTGGGCAGGTAGGTGAG | 20 |
| 1-1 | | |
| HER2 V-type probe | GCAGGAAGGACAGGCTGGCAAAAAAAAAAATGGCTACTACACTCTT | 21 |
| 2-1 | | |
| HER2 RNA site 2 | TCGAAGCCTCACAGAGATCTTGAAAGGAGGGGTCTTGATC | 22 |
| HER2 V-type probe | CGCTAATAGTCGATAAAAAAAAAAAAGATCTCTGTGAGGCTTCGA | 23 |
| 1-2 | | |
| HER2 V-type probe | GATCAAGACCCCTCCTTTCAAAAAAAAAAATGGCTACTACACTCTT | 24 |
| 2-2 | | |
| HER2 RNA site 3 | ACCTTTCTACGGACGTGGGATCCTGCACCCTCGTCTGCCC | 25 |
| HER2 V-type probe | CGCTAATAGTCGATAAAAAAAAAAATCCCACGTCCGTAGAAAGGT | 26 |
| 1-3 | | |
| HER2 V-type probe | GGGCAGACGAGGGTGCAGGAAAAAAAAAAATGGCTACTACACTCTT | 27 |
| 2-3 | | |
| HER2 RNA site 4 | TTCCAGAACCTGCAAGTAATCCGGGGACGAATTCTGCACA | 28 |
| HER2 V-type probe | CGCTAATAGTCGATAAAAAAAAAAAATTACTTGCAGGTTCTGGAA | 29 |
| 1-4 | | |
| HER2 V-type probe | TGTGCAGAATTCGTCCCCGGAAAAAAAAAATGGCTACTACACTCTT | 30 |
| 2-4 | | |
| HER2 RNA site 5 | AGGGCCCACCCAGTGTGTCAACTGCAGCCAGTTCCTTCGG | 31 |
| HER2 V-type probe | CGCTAATAGTCGATAAAAAAAAAAATGACACACTGGGTGGGCCCT | 32 |
| 1-5 | | |
| HER2 V-type probe | CCGAAGGAACTGGCTGCAGTAAAAAAAAAATGGCTACTACACTCTT | 33 |
| 2-5 | | |
| UBC RNA site 1 | CAGCCGGGATTTGGGTCGCAGTTCTTGTTTGTGGATCGCT | 34 |
| UBC V-type probe 1-1 | CGCTAATGGCTCCACAAAAAAAAAATGCGACCCAAATCCCGGCTG | 35 |
| UBC V-type probe 2-1 | AGCGATCCACAAACAAGAACAAAAAAAAAACAGACGCAACACTCTT | 36 |
| UBC RNA site 2 | GGGATGCAGATCTTCGTGAAGACCCTGACTGGTAAGACCA | 37 |
| UBC V-type probe 1-2 | CGCTAATGGCTCCACAAAAAAAAAATTCACGAAGATCTGCATCCC | 38 |
| UBC V-type probe 2-2 | TGGTCTTACCAGTCAGGGTCAAAAAAAAAACAGACGCAACACTCTT | 39 |
| UBC RNA site 3 | CAGAAAGAGTCCACTCTGCACTTGGTCCTGCGCTTGAGGG | 40 |
| UBC V-type probe 1-3 | CGCTAATGGCTCCACAAAAAAAAAATGCAGAGTGGACTCTTTCTG | 41 |
| UBC V-type probe 2-3 | CCCTCAAGCGCAGGACCAAGAAAAAAAAAACAGACGCAACACTCTT | 42 |
| UBC RNA site 4 | TGGGCGCACCCTGTCTGACTACAACATCCAGAAAGAGTCC | 43 |
| UBC V-type probe 1-4 | CGCTAATGGCTCCACAAAAAAAAAAAGTCAGACAGGGTGCGCCCA | 44 |
| UBC V-type probe 2-4 | GGACTCTTTCTGGATGTTGTAAAAAAAAAACAGACGCAACACTCTT | 45 |
| UBC RNA site 5 | GTGAAGACACTCACTGGCAAGACCATCACCCTTGAGGTCG | 46 |
| UBC V-type probe 1-5 | CGCTAATGGCTCCACAAAAAAAAAATTGCCAGTGAGTGTCTTCAC | 47 |
| UBC V-type probe 2-5 | CGACCTCAAGGGTGATGGTCAAAAAAAAAACAGACGCAACACTCTT | 48 |
| dapB RNA site 1 | AGAATCATGGCGTATCTGAAGCGTTTGGCCATCCATGCCG | 49 |
| dapB V-type probe 1-1 | CGCTAATAGCGATTAAAAAAAAAAATTCAGATACGCCATGATTCT | 50 |
| dapB V-type probe 2-1 | CGGCATGGATGGCCAAACGCAAAAAAAAAACAGCGCGAACACTCTT | 51 |
| dapB RNA site 2 | AGTTCCGCTTGGTGCGTCAAGCTTCTGGTCATGATGAAGC | 52 |
| dapB V-type probe 1-2 | CGCTAATAGCGATTAAAAAAAAAAATTGACGCACCAAGCGGAACT | 53 |
| dapB V-type probe 2-2 | GCTTCATCATGACCAGAAGCAAAAAAAAAACAGCGCGAACACTCTT | 54 |
| dapB RNA site 3 | CTTCTGAGAAACCGGTTGTTCCGACAACTGGACGGACTCC | 55 |
| dapB V-type probe 1-3 | CGCTAATAGCGATTAAAAAAAAAAAAACAACCGGTTTCTCAGAAG | 56 |
| dapB V-type probe 2-3 | GGAGTCCGTCCAGTTGTCGGAAAAAAAAAACAGCGCGAACACTCTT | 57 |
| dapB RNA site 4 | TGTGGTGTTCGTTCTGCCAATTTAACAGCTTCCTGCCCCA | 58 |
| dapB V-type probe 1-4 | CGCTAATAGCGATTAAAAAAAAAAATTGGCAGAACGAACACCACA | 59 |
| dapB V-type probe 2-4 | TGGGGCAGGAAGCTGTTAAAAAAAAAAAAACAGCGCGAACACTCTT | 60 |
| dapB RNA site 5 | GATCAGTCCCGGAAGACGGACGCTGTGCAAGCGAATACCG | 61 |
| dapB V-type probe 1-5 | CGCTAATAGCGATTAAAAAAAAAAATCCGTCTTCCGGGACTGATC | 62 |
| dapB V-type probe 2-5 | CGGTATTCGCTTGCACAGCGAAAAAAAAAACAGCGCGAACACTCTT | 63 |
| UBC detection probe | TGCGTCTATTTAGTGGAGCC | 64 |
| UBC padlock probe | ATTAGCGGTCCGTCTAGGAGAGTAGTACAGCAGCCGTCAAGAGTGTT | 65 |
| | GCGTCTATTTAGTGGAGCC | |
| dapB detection probe | TCGCGCTTGGTATAATCGCT | 66 |
| dapB padlock probe | ATTAGCGGTCCGTCTAGGAGAGTAGTACAGCAGCCGTCAAGAGTGTT | 67 |
| | CGCGCTTGGTATAATCGCT | |
| HER2 detection probe | AGTAGCCGTGACTATCGACT | 1 |
| HER2 padlock probe | ATTAGCGGTCCGTCTAGGAGAGTAGTACAGCAGCCGTCAAGAGTGT | 2 |
| | AGTAGCCGTGACTATCGACT | |

The results of this experiment were shown in FIG. 2 and Table 10 (in which the red fluorescence was HER2, the silver fluorescence was UBC, and the indigo fluorescence was dapB): dapB was a negative control gene, that was, theoretically there should be no detection, and its detection amount in this method was only 0.02, which was consistent with reality; UBC was a medium-highly expressed housekeeping gene, and HER2 was a SKBR3-specific high-expression gene, and the detection amounts of these two genes in this method were in line with expectations.

TABLE 10
Number of fluorescent signals

| | dapB | UBC | HER2 |
|---|---|---|---|
| Number of signals | 0.02 | 72.12 | 98.39 |

In summary, this method had the ability to perform in-situ multiplex detection of two or more RNAs, and could detect the gene expression levels.

### Example 3: Detection Length of V-Type Probes

In order to explore the minimum target RNA length that the V-type probes could detect, V-type probes were designed in this example, so that their single hybridization lengths with the target RNA sequence (detecting HER2 on SKBR3) were 10 nt, 12 nt, 15 nt, and 20 nt. The experimental procedures were the same as those described in Example 1.1, and the specific probes and reagents used were shown in Table 11 below.

TABLE 11
Probe sequences

| Description | Sequence | SEQ ID NO: |
|---|---|---|
| HER2 detection probe | AGTAGCCGTGACTATCGACT | 1 |
| HER2 padlock probe | ATTAGCGGTCCGTCTAGGAGAGTAGTACAGCAGCCGTCAAGAG | 2 |
| | TGTAGTAGCCGTGACTATCGACT | |
| HER2 RNA site 1 | TGCCCACCAATGCCAGCCTG | 68 |
| V-type probe 10nt1-1 | CGCTAATAGTCGATAAAAAAAAAAATTGGTGGGCA | 69 |
| V-type probe 10nt2-1 | CAGGCTGGCAAAAAAAAAAATGGCTACTACACTCTT | 70 |
| RNA site 2 | ACAGAGATCTTGAAAGGAGG | 71 |
| V-type probe 10nt1-2 | CGCTAATAGTCGATAAAAAAAAAAAAGATCTCTGT | 72 |
| V-type probe 10nt2-2 | CCTCCTTTCAAAAAAAAAAATGGCTACTACACTCTT | 73 |
| RNA site 3 | GGACGTGGGATCCTGCACCC | 74 |
| V-type probe 10nt1-3 | CGCTAATAGTCGATAAAAAAAAAAATCCCACGTCC | 75 |
| V-type probe 10nt2-3 | GGGTGCAGGAAAAAAAAAAATGGCTACTACACTCTT | 76 |
| RNA site 1 | CCTGCCCACCAATGCCAGCCTGTC | 77 |
| V-type probe 12nt1-1 | CGCTAATAGTCGATAAAAAAAAAAATTGGTGGGCAGG | 78 |
| V-type probe 12nt2-1 | GACAGGCTGGCAAAAAAAAAAATGGCTACTACACTCTT | 79 |
| RNA site 2 | TCACAGAGATCTTGAAAGGAGGGG | 80 |
| V-type probe 12nt1-2 | CGCTAATAGTCGATAAAAAAAAAAAAGATCTCTGTGA | 81 |
| V-type probe 12nt2-2 | CCCCTCCTTTCAAAAAAAAAAATGGCTACTACACTCTT | 82 |
| RNA site 3 | ACGGACGTGGGATCCTGCACCCTC | 83 |
| V-type probe 12nt1-3 | CGCTAATAGTCGATAAAAAAAAAAATCCCACGTCCGT | 84 |
| V-type probe 12nt2-3 | GAGGGTGCAGGAAAAAAAAAAATGGCTACTACACTCTT | 85 |
| RNA site 1 | CTACCTGCCCACCAATGCCAGCCTGTCCTT | 86 |
| V-type probe 15nt1-1 | CGCTAATAGTCGATAAAAAAAAAAATTGGTGGGCAGGTAG | 87 |
| V-type probe 15nt2-1 | AAGGACAGGCTGGCAAAAAAAAAAATGGCTACTACACTCTT | 88 |
| RNA site 2 | GCCTCACAGAGATCTTGAAAGGAGGGGTCT | 89 |
| V-type probe 15nt1-2 | CGCTAATAGTCGATAAAAAAAAAAAAGATCTCTGTGAGGC | 90 |
| V-type probe 15nt2-2 | AGACCCCTCCTTTCAAAAAAAAAAATGGCTACTACACTCTT | 91 |
| RNA site 3 | TCTACGGACGTGGGATCCTGCACCCTCGTC | 92 |
| V-type probe 15nt1-3 | CGCTAATAGTCGATAAAAAAAAAAATCCCACGTCCGTAGA | 93 |
| V-type probe 15nt2-3 | GACGAGGGTGCAGGAAAAAAAAAAATGGCTACTACACTCTT | 94 |
| RNA site 1 | CTCACCTACCTGCCCACCAATGCCAGCCTGTCCTTCCTGC | 19 |
| V-type probe 20nt1-1 | CGCTAATAGTCGATAAAAAAAAAAATTGGTGGGCAGGTAGGTG | 20 |
| | AG | |
| V-type probe 20nt2-1 | GCAGGAAGGACAGGCTGGCAAAAAAAAAAATGGCTACTACACT | 21 |
| | CTT | |
| RNA site 2 | TCGAAGCCTCACAGAGATCTTGAAAGGAGGGGTCTTGATC | 22 |
| V-type probe 20nt1-2 | CGCTAATAGTCGATAAAAAAAAAAAAGATCTCTGTGAGGCTTC | 23 |
| | GA | |
| V-type probe 20nt2-2 | GATCAAGACCCCTCCTTTCAAAAAAAAAAATGGCTACTACACTC | 24 |
| | TT | |
| RNA site 3 | ACCTTTCTACGGACGTGGGATCCTGCACCCTCGTCTGCCC | 25 |
| V-type probe 20nt1-3 | CGCTAATAGTCGATAAAAAAAAAAATCCCACGTCCGTAGAAAG | 26 |
| | GT | |
| V-type probe 20nt2-3 | GGGCAGACGAGGGTGCAGGAAAAAAAAAAATGGCTACTACAC | 27 |
| | TCTT | |

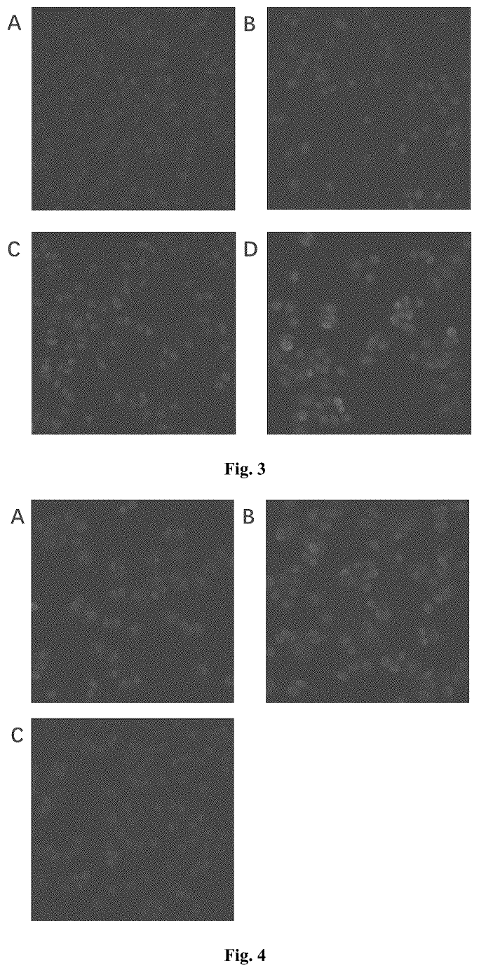

The results obtained in this experiment were shown in FIG. 3 and Table 12 (FIG. 3A showed 10 nt, FIG. 3B showed 12 nt, FIG. 3C showed 15 nt, FIG. 3D showed 20 nt): 10 nt basically had no signal point detected, and its value was 0; 12 nt had signal points detected, and its value was 1.30; 15 nt had signal points detected, and its value was 34.52; 20 nt had signal points detected, and its value was 116.95. The detection results showed that this method had the potential to detect RNA with extremely short sequence, such as microRNA.

TABLE 12
Number of fluorescent signals

| | 10 nt | 12 nt | 15 nt | 20 nt |
|---|---|---|---|---|
| HER2 | 0.00 | 1.30 | 34.52 | 116.95 |

### Example 4: Exploration of V-Type Probe Spacer Sequence

In order to explore the length of V-type probe spacer sequence, V-type probes 1 and 2 were designed in this example, respectively, so that the spacer sequences between them and the hybridization section of padlock probe were 0, 5, and 10 nt (detecting HER2 on SKBR3), respectively. The experimental steps and reagents used were the same as those described in Example 1.1, and the specific probes used were shown in Table 13 below.

TABLE 13
Probe sequences

| Description | Sequence | SEQ ID NO: |
|---|---|---|
| HER2 detection probe | AGTAGCCGTGACTATCGACT | 1 |
| HER2 padlock probe | ATTAGCGGTCCGTCTAGGAGAGTAGTACAGCAGCCGTCAAGAGTGT | 2 |
| | AGTAGCCGTGACTATCGACT | |
| RNA site 1 | CTCACCTACCTGCCCACCAATGCCAGCCTGTCCTTCCTGC | 19 |
| V-type probe Ont1-1 | CGCTAATAGTCGATAGTAAAAAAAAAATTGGTGGGCAGGTAGGTGA | 95 |
| | G | |
| V-type probe Ont2-1 | GCAGGAAGGACAGGCTGGCAAAAAAAAAAACACGGCTACTACACTC | 96 |
| | TT | |
| RNA site 2 | TCGAAGCCTCACAGAGATCTTGAAAGGAGGGGTCTTGATC | 22 |
| V-type probe Ont1-2 | CGCTAATAGTCGATAGTAAAAAAAAAAAGATCTCTGTGAGGCTTCG | 97 |
| | A | |
| V-type probe Ont2-2 | GATCAAGACCCCTCCTTTCAAAAAAAAAAACACGGCTACTACACTCT | 98 |
| | T | |
| RNA site 3 | ACCTTTCTACGGACGTGGGATCCTGCACCCTCGTCTGCCC | 25 |
| V-type probe Ont1-3 | CGCTAATAGTCGATAGTAAAAAAAAAATCCCACGTCCGTAGAAAGG | 99 |
| | T | |
| V-type probe Ont2-3 | GGGCAGACGAGGGTGCAGGAAAAAAAAAAACACGGCTACTACACTC | 100 |
| | TT | |
| RNA site 1 | CTCACCTACCTGCCCACCAATGCCAGCCTGTCCTTCCTGC | 19 |
| V-type probe 5nt1-1 | CGCTAATAGTCGATAAAAAAAAAAATTGGTGGGCAGGTAGGTGAG | 20 |
| V-type probe 5nt2-1 | GCAGGAAGGACAGGCTGGCAAAAAAAAAAATGGCTACTACACTCTT | 21 |
| RNA site 2 | TCGAAGCCTCACAGAGATCTTGAAAGGAGGGGTCTTGATC | 22 |
| V-type probe 5nt1-2 | CGCTAATAGTCGATAAAAAAAAAAAAGATCTCTGTGAGGCTTCGA | 23 |
| V-type probe 5nt2-2 | GATCAAGACCCCTCCTTTCAAAAAAAAAAATGGCTACTACACTCTT | 24 |
| RNA site 3 | ACCTTTCTACGGACGTGGGATCCTGCACCCTCGTCTGCCC | 25 |
| V-type probe 5nt1-3 | CGCTAATAGTCGATAAAAAAAAAAATCCCACGTCCGTAGAAAGGT | 26 |
| V-type probe 5nt2-3 | GGGCAGACGAGGGTGCAGGAAAAAAAAAAATGGCTACTACACTCTT | 27 |
| RNA site 1 | CTCACCTACCTGCCCACCAATGCCAGCCTGTCCTTCCTGC | 19 |
| V-type probe 10nt1-1 | CGCTAATAGTCGCGCAAAAAAAAAATTGGTGGGCAGGTAGGTGAG | 10 |
| V-type probe 10nt2-1 | GCAGGAAGGACAGGCTGGCAAAAAAAAAAATAACTACTACACTCTT | 102 |
| RNA site 2 | TCGAAGCCTCACAGAGATCTTGAAAGGAGGGGTCTTGATC | 22 |
| V-type probe 10nt1-2 | CGCTAATAGTCGCGCAAAAAAAAAAAGATCTCTGTGAGGCTTCGA | 103 |
| V-type probe 10nt2-2 | GATCAAGACCCCTCCTTTCAAAAAAAAAAATAACTACTACACTCTT | 104 |
| RNA site 3 | ACCTTTCTACGGACGTGGGATCCTGCACCCTCGTCTGCCC | 25 |
| V-type probe 10nt1-3 | CGCTAATAGTCGCGCAAAAAAAAAATCCCACGTCCGTAGAAAGGT | 105 |
| V-type probe 10nt2-3 | GGGCAGACGAGGGTGCAGGAAAAAAAAAAATAACTACTACACTCTT | 106 |

The comparative experimental results of the V-type probe spacer sequence lengths were shown in FIG. 4 and Table 14 below, in which when the spacer sequence was 5 nt, HER2 had the highest detection amount of 114.57 on SKBR3, and when the spacer sequence was extended to 10 nt, the detection amount was only 0.23. The reason for this result might not only be related to the expansion of the spacer sequence, but also the shortening of the length of hybridization sequence between the V-type probe and the padlock probe caused by the expansion of the spacer sequence (because the sum of the length of the spacer sequence and the length of hybridization sequence between the V-type probe and the padlock probe was the total length of the detection probe sequence, which was a constant value).

TABLE 14
Number of fluorescent signals

| | 0 nt | 5 nt | 10 nt |
|---|---|---|---|
| HER2 | 46.25 | 114.57 | 0.23 |

This result showed that under the current scheme of this method, the V-type probe spacer sequence lengths of 0 to 10 nt all had detection signals, and 5 nt showed the best result. Therefore, we selected 5 nt as the spacer sequence length in routine experiments.

### Example 5: Exploration of Hybridization Length of V-Type Probe and Padlock Probe

In order to explore the effect of hybridization length of the V-type probe and the padlock probe, different V-type probes 1 and 2 were designed in this example, respectively, to explore the effect of hybridization lengths of them and the padlock probe (detecting HER2 on SKBR3). The experimental steps and reagents used were the same as those described in Example 1.1.

First, the length of the hybridization sequence between V-type probe 2 and the padlock probe was fixed (making V-type probe 2 to have hybridization of 9 bp with the backbone sequence of the padlock probe, and hybridization of 8 bp with the detection probe sequence of the padlock probe), and the length of the hybridization sequence between V-type probe 1 and the padlock probe was changed. Wherein, the first V-type probe 1 had hybridization of 8 bp with the backbone sequence of the padlock probe, and hybridization of 7 bp with the detection probe of the padlock probe; the second V-type probe 1 had hybridization of 7 bp with the backbone sequence of the padlock probe, and hybridization of 6 bp with the detection probe of the padlock probe; the third V-type probe 1 had hybridization of 6 bp with the backbone sequence of the padlock probe, and hybridization of 6 pb with the detection probe of the padlock probe; and the above 4 kinds of probes were designed for 5 RNA sites of HER2, respectively, and the specific probe names and sequences were shown in Table 15.

TABLE 15
Probe sequences

| Description | Sequence | SEQ ID NO: |
|---|---|---|
| HER2 detection | AGTAGCCGTGACTATCGACT | 1 |
| probe | | |
| Padlock probe | ATTAGCGTGTCCGTCTAGGAGAGTAGTACAGCAGCCGTCAAGAGT | 107 |
| 8 + 7 − 9 + 8 | GTCAGTAGCCGTGACTATCGACT | |
| Padlock probe | ATTAGCGGTCCGTCTAGGAGAGTAGTACAGCAGCCGTCAAGAGTG | 108 |
| 7 + 6 − 9 + 8 | TCAGTAGCCGTGACTATCGACT | |
| Padlock probe | ATTAGCGTCCGTCTAGGAGAGTAGTACAGCAGCCGTCAAGAGTGT | 109 |
| 6 + 6 − 9 + 8 | CAGTAGCCGTGACTATCGACT | |
| RNA site 1 | CTACCTGCCCACCAATGCCAGCCTGTCCTT | 110 |
| V-type probe | ACGCTAATAGTCGATCAAAAAAAAAATTGGTGGGCAGGTAG | 111 |
| 8 + 7 − 1 − 1 | | |
| V-type probe | CGCTAATAGTCGAGCAAAAAAAAAATTGGTGGGCAGGTAG | 112 |
| 7 + 6 − 1 − 1 | | |
| V-type probe | GCTAATAGTCGAGCAAAAAAAAAATTGGTGGGCAGGTAG | 113 |
| 6 + 6 − 1 − 1 | | |
| V-type probe | AAGGACAGGCTGGCAAAAAAAAAAACGGCTACTGACACTCTT | 114 |
| 9 + 8 − 2 − 1 | | |
| RNA site 2 | AGCCTCACAGAGATCTTGAAAGGAGGGGTCTT | 115 |
| V-type probe | ACGCTAATAGTCGATCAAAAAAAAAAAGATCTCTGTGAGGCT | 116 |
| 8 + 7 − 1 − 2 | | |
| V-type probe | CGCTAATAGTCGAGCAAAAAAAAAAAGATCTCTGTGAGGCT | 117 |
| 7 + 6 − 1 − 2 | | |
| V-type probe | GCTAATAGTCGAGCAAAAAAAAAAAGATCTCTGTGAGGCT | 118 |
| 6 + 6 − 1 − 2 | | |
| V-type probe | AAGACCCCTCCTTTCAAAAAAAAAAACGGCTACTGACACTCTT | 119 |
| 9 + 8 − 2 − 2 | | |
| RNA site 3 | TTCTACGGACGTGGGATCCTGCACCCTCGTCT | 120 |
| V-type probe | ACGCTAATAGTCGATCAAAAAAAAAATCCCACGTCCGTAGAA | 121 |
| 8 + 7 − 1 − 3 | | |
| V-type probe | CGCTAATAGTCGAGCAAAAAAAAAATCCCACGTCCGTAGAA | 122 |
| 7 + 6 − 1 − 3 | | |
| V-type probe | GCTAATAGTCGAGCAAAAAAAAAATCCCACGTCCGTAGAA | 123 |
| 6 + 6 − 1 − 3 | | |
| V-type probe | AGACGAGGGTGCAGGAAAAAAAAAAACGGCTACTGACACTCTT | 124 |
| 9 + 8 − 2 − 3 | | |
| RNA site 4 | AGAACCTGCAAGTAATCCGGGGACGAATTCTG | 125 |
| V-type probe | ACGCTAATAGTCGATCAAAAAAAAAAATTACTTGCAGGTTCT | 126 |
| 8 + 7 − 1 − 4 | | |
| V-type probe | CGCTAATAGTCGAGCAAAAAAAAAAATTACTTGCAGGTTCT | 127 |
| 7 + 6 − 1 − 4 | | |
| V-type probe | GCTAATAGTCGAGCAAAAAAAAAAATTACTTGCAGGTTCT | 128 |
| 6 + 6 − 1 − 4 | | |
| V-type probe | CAGAATTCGTCCCCGGAAAAAAAAAACGGCTACTGACACTCTT | 129 |
| 9 + 8 − 2 − 4 | | |
| RNA site 5 | CCCACCCAGTGTGTCAACTGCAGCCAGTTCCT | 130 |
| V-type probe | ACGCTAATAGTCGATCAAAAAAAAAATGACACACTGGGTGGG | 131 |
| 8 + 7 − 1 − 5 | | |
| V-type probe | CGCTAATAGTCGAGCAAAAAAAAAATGACACACTGGGTGGG | 132 |
| 7 + 6 − 1 − 5 | | |
| V-type probe | GCTAATAGTCGAGCAAAAAAAAAATGACACACTGGGTGGG | 133 |
| 6 + 6 − 1 − 5 | | |
| V-type probe | AGGAACTGGCTGCAGTAAAAAAAAAACGGCTACTGACACTCTT | 134 |
| 9 + 8 − 2 − 5 | | |

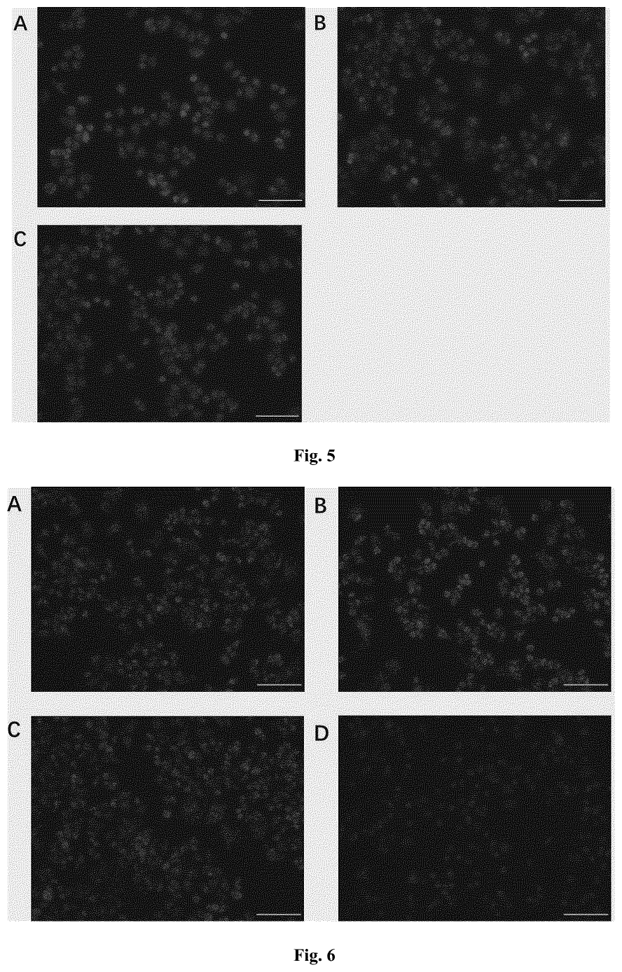

The experimental results were shown in FIG. 5 and Table 16. In the case that the V-type probe 2 had hybridization of 9 bp with the backbone sequence of the padlock probe and hybridization of 8 bp with the detection probe sequence of the padlock probe, when the V-type probe 1 had hybridization of 8 bp with the backbone sequence of the padlock probe and hybridization of 7 bp with the detection probe of the padlock probe, the extracellular noise was lower and the overall result was the most ideal.

TABLE 16
Number of fluorescent signals

SKBR3

| | HER2 | Cells |
|---|---|---|
| 8 + 7 | 63.01 | 663.00 |
| 7 + 6 | 56.63 | 1027.00 |
| 6 + 6 | 49.15 | 872.00 |

Then, the length of the hybridization sequence between V-type probe 1 and the padlock probe was fixed (making the V-type probe 1 had hybridization of 7 bp with the backbone sequence of the padlock probe, and hybridization of 6 bp with the detection probe sequence of the padlock probe), and the length of the hybridization sequence between V-type probe 2 and the padlock probe was changed. Wherein, the first V-type probe 2 had hybridization of 9 bp with the backbone sequence of the padlock probe and hybridization of 8 bp with the detection probe of the padlock probe; the second V-type probe 2 had hybridization of 8 bp with the backbone sequence of the padlock probe, and hybridization of 7 bp with the detection probe of the padlock probe; the third V-type probe 2 had hybridization of 7 bp with the backbone sequence of the padlock probe, and hybridization of 6 bp with the detection probe of the padlock probe; the fourth V-type probe 2 had hybridization of 6 bp with the backbone sequence of the padlock probe and hybridization of 6 bp with the detection probe of the padlock probe. The above probes were designed respectively for the five RNA sites of HER2. The specific probe names and sequences were shown in Table 17 below.

TABLE 17
Probe sequences

| Description | Sequence | SEQ ID NO: |
|---|---|---|
| HER2 detection | AGTAGCCGTGACTATCGACT | 1 |
| probe | | |
| Padlock probe | ATTAGCGGTCCGTCTAGGAGAGTAGTACAGCAGCCGTCA | 135 |
| 7 + 6 − 9 + 8 | AGAGTGTCAGTAGCCGTGACTATCGACT | |
| Padlock probe | ATTAGCGGTCCGTCTAGGAGAGTAGTACAGCAGCCGTCA | 2 |
| 7 + 6 − 8 + 7 | AGAGTGTAGTAGCCGTGACTATCGACT | |
| Padlock probe | ATTAGCGGTCCGTCTAGGAGAGTAGTACAGCAGCCGTCA | 136 |
| 7 + 6 − 7 + 6 | AGAGTGAGTAGCCGTGACTATCGACT | |
| Padlock probe | ATTAGCGGTCCGTCTAGGAGAGTAGTACAGCAGCCGTCA | 137 |
| 7 + 6 − 6 + 6 | AGAGTAGTAGCCGTGACTATCGACT | |
| RNA site 1 | CTACCTGCCCACCAATGCCAGCCTGTCCTT | 138 |
| V-type probe | CGCTAATAGTCGAGCAAAAAAAAAATTGGTGGGCAGGTA | 139 |
| 7 + 6 − 1 − 1 | G | |
| V-type probe | AAGGACAGGCTGGCAAAAAAAAAAACGGCTACTGACACT | 140 |
| 9 + 8 − 2 − 1 | CTT | |
| V-type probe | AAGGACAGGCTGGCAAAAAAAAAAATGGCTACTACACTC | 141 |
| 8 + 7 − 2 − 1 | TT | |
| V-type probe | AAGGACAGGCTGGCAAAAAAAAAAATAGCTACTCACTCT | 142 |
| 7 + 6 − 2 − 1 | T | |
| V-type probe | AAGGACAGGCTGGCAAAAAAAAAAATAGCTACTACTCTT | 143 |
| 6 + 6 − 2 − 1 | | |
| RNA site 2 | AGCCTCACAGAGATCTTGAAAGGAGGGGTCTT | 144 |
| V-type probe | CGCTAATAGTCGAGCAAAAAAAAAAAGATCTCTGTGAGG | 145 |
| 7 + 6 − 1 − 2 | CT | |
| V-type probe | AAGACCCCTCCTTTCAAAAAAAAAAACGGCTACTGACAC | 146 |
| 9 + 8 − 2 − 2 | TCTT | |
| V-type probe | AAGACCCCTCCTTTCAAAAAAAAAAATGGCTACTACACT | 147 |
| 8 + 7 − 2 − 2 | CTT | |
| V-type probe | AAGACCCCTCCTTTCAAAAAAAAAAATAGCTACTCACTCT | 148 |
| 7 + 6 − 2 − 2 | T | |
| V-type probe | AAGACCCCTCCTTTCAAAAAAAAAAATAGCTACTACTCTT | 149 |
| 6 + 6 − 2 − 2 | | |
| RNA site 3 | TTCTACGGACGTGGGATCCTGCACCCTCGTCT | 150 |
| V-type probe | CGCTAATAGTCGAGCAAAAAAAAAATCCCACGTCCGTAG | 151 |
| 7 + 6 − 1 − 3 | AA | |
| V-type probe | AGACGAGGGTGCAGGAAAAAAAAAAACGGCTACTGACA | 152 |
| 9 + 8 − 2 − 3 | CTCTT | |
| V-type probe | AGACGAGGGTGCAGGAAAAAAAAAAATGGCTACTACACT | 153 |
| 8 + 7 − 2 − 3 | CTT | |
| V-type probe | AGACGAGGGTGCAGGAAAAAAAAAAATAGCTACTCACTC | 154 |
| 7 + 6 − 2 − 3 | TT | |
| V-type probe | AGACGAGGGTGCAGGAAAAAAAAAAATAGCTACTACTCT | 155 |
| 6 + 6 − 2 − 3 | T | |
| RNA site 4 | AGAACCTGCAAGTAATCCGGGGACGAATTCTG | 156 |
| V-type probe | CGCTAATAGTCGAGCAAAAAAAAAAATTACTTGCAGGTT | 157 |
| 7 + 6 − 1 − 4 | CT | |
| V-type probe | CAGAATTCGTCCCCGGAAAAAAAAAACGGCTACTGACAC | 158 |
| 9 + 8 − 2 − 4 | TCTT | |
| V-type probe | CAGAATTCGTCCCCGGAAAAAAAAAATGGCTACTACACT | 159 |
| 8 + 7 − 2 − 4 | CTT | |
| V-type probe | CAGAATTCGTCCCCGGAAAAAAAAAATAGCTACTCACTC | 160 |
| 7 + 6 − 2 − 4 | TT | |
| V-type probe | CAGAATTCGTCCCCGGAAAAAAAAAATAGCTACTACTCT | 161 |
| 6 + 6 − 2 − 4 | T | |
| RNA site 5 | CCCACCCAGTGTGTCAACTGCAGCCAGTTCCT | 162 |
| V-type probe | CGCTAATAGTCGAGCAAAAAAAAAATGACACACTGGGTG | 163 |
| 7 + 6 − 1 − 5 | GG | |
| V-type probe | AGGAACTGGCTGCAGTAAAAAAAAAACGGCTACTGACAC | 164 |
| 9 + 8 − 2 − 5 | TCTT | |
| V-type probe | AGGAACTGGCTGCAGTAAAAAAAAAATGGCTACTACACT | 165 |
| 8 + 7 − 2 − 5 | CTT | |
| V-type probe | AGGAACTGGCTGCAGTAAAAAAAAAATAGCTACTCACTC | 166 |
| 7 + 6 − 2 − 5 | TT | |
| V-type probe | AGGAACTGGCTGCAGTAAAAAAAAAATAGCTACTACTCT | 167 |
| 6 + 6 − 2 − 5 | T | |

The experimental results were shown in FIG. 6 and Table 18. In the case that the V-type probe 1 had hybridization of 7 bp with the backbone sequence of the padlock probe and hybridization of 6 bp with the detection probe sequence of the padlock probe, when the V-type probe 2 had hybridization of 8 bp with the backbone sequence of the padlock probe and hybridization of 7 bp with the detection probe of the padlock probe, the extracellular noise was low and the overall result was the most ideal.

TABLE 18
Number of fluorescent signals

| | HER2 | cells |
|---|---|---|
| 9 + 8 | 33.25 | 900.00 |
| 8 + 7 | 38.56 | 861.00 |
| 7 + 6 | 22.78 | 1387.00 |
| 6 + 6 | 1.03 | 962.00 |

According to the hybridization length obtained in the above experiment, the length of the hybridization sequence between V-type probe 2 and the padlock probe was fixed (making the V-type probe 2 to have hybridization of 8 bp with the backbone sequence of the padlock probe, and hybridization of 7 bp with the detection probe sequence of the padlock probe), and the length of the hybridization sequence between V-type probe 1 and the padlock probe was changed. Wherein, the first V-type probe 1 had hybridization of 7 bp with the backbone sequence of the padlock probe and hybridization of 8 bp with the detection probe of the padlock probe; the second V-type probe 2 had hybridization of 7 bp with the backbone sequence of the padlock probe, and hybridization of 7 bp with the detection probe of the padlock probe; the third V-type probe 2 had hybridization of 6 bp with the backbone sequence of the padlock probe, and hybridization of 7 bp with the detection probe of the padlock probe. The above 4 probes were designed respectively for the five RNA sites of HER2. The specific probe names and sequences were shown in Table 19 below.

TABLE 19
Probe sequences

| Description | Sequence | SEQ ID NO: |
|---|---|---|
| HER2 detection | AGTAGCCGTGACTATCGACT | 1 |
| probe | | |
| Padlock probe | ATTAGCGGTCCGTCTAGGAGAGTAGTACAGCAGCCGTCAA | 2 |
| 7 + 8 − 8 + 7 | GAGTGTAGTAGCCGTGACTATCGACT | |
| Padlock probe | ATTAGCGGTCCGTCTAGGAGAGTAGTACAGCAGCCGTCAA | 2 |
| 7 + 7 − 8 + 7 | GAGTGTAGTAGCCGTGACTATCGACT | |
| Padlock probe | ATTAGCGTCCGTCTAGGAGAGTAGTACAGCAGCCGTCAAG | 168 |
| 6 + 7 − 8 + 7 | AGTGTAGTAGCCGTGACTATCGACT | |
| RNA site 1 | CTACCTGCCCACCAATGCCAGCCTGTCCTT | 169 |
| V-type probe | CGCTAATAGTCGATAAAAAAAAAAATTGGTGGGCAGGTAG | 170 |
| 7 + 8 − 1 − 1 | | |
| V-type probe | CGCTAATAGTCGATCAAAAAAAAAATTGGTGGGCAGGTAG | 171 |
| 7 + 7 − 1 − 1 | | |
| V-type probe | GCTAATAGTCGATCAAAAAAAAAATTGGTGGGCAGGTAG | 172 |
| 6 + 7 − 1 − 1 | | |
| V-type probe | AAGGACAGGCTGGCAAAAAAAAAAATGGCTACTACACTCT | 173 |
| 8 + 7 − 2 − 1 | T | |
| RNA site 2 | AGCCTCACAGAGATCTTGAAAGGAGGGGTCTT | 174 |
| V-type probe | CGCTAATAGTCGATAAAAAAAAAAAAGATCTCTGTGAGGC | 175 |
| 7 + 8 − 1 − 2 | T | |
| V-type probe | CGCTAATAGTCGATCAAAAAAAAAAAGATCTCTGTGAGGC | 176 |
| 7 + 7 − 1 − 2 | T | |
| V-type probe | GCTAATAGTCGATCAAAAAAAAAAAGATCTCTGTGAGGCT | 177 |
| 6 + 7 − 1 − 2 | | |
| V-type probe | AAGACCCCTCCTTTCAAAAAAAAAAATGGCTACTACACTCT | 178 |
| 8 + 7 − 2 − 2 | T | |
| RNA site 3 | TTCTACGGACGTGGGATCCTGCACCCTCGTCT | 179 |
| V-type probe | CGCTAATAGTCGATAAAAAAAAAAATCCCACGTCCGTAGA | 180 |
| 7 + 8 − 1 − 3 | A | |
| V-type probe | CGCTAATAGTCGATCAAAAAAAAAATCCCACGTCCGTAGA | 181 |
| 7 + 7 − 1 − 3 | A | |
| V-type probe | GCTAATAGTCGATCAAAAAAAAAATCCCACGTCCGTAGAA | 182 |
| 6 + 7 − 1 − 3 | | |
| V-type probe | AGACGAGGGTGCAGGAAAAAAAAAAATGGCTACTACACTC | 183 |
| 8 + 7 − 2 − 3 | TT | |
| RNA site 4 | AGAACCTGCAAGTAATCCGGGGACGAATTCTG | 184 |
| V-type probe | CGCTAATAGTCGATAAAAAAAAAAAATTACTTGCAGGTTCT | 185 |
| 7 + 8 − 1 − 4 | | |
| V-type probe | CGCTAATAGTCGATCAAAAAAAAAAATTACTTGCAGGTTCT | 186 |
| 7 + 7 − 1 − 4 | | |
| V-type probe | GCTAATAGTCGATCAAAAAAAAAAATTACTTGCAGGTTCT | 187 |
| 6 + 7 − 1 − 4 | | |
| V-type probe | CAGAATTCGTCCCCGGAAAAAAAAAATGGCTACTACACTCT | 188 |
| 8 + 7 − 2 − 4 | T | |
| RNA site 5 | CCCACCCAGTGTGTCAACTGCAGCCAGTTCCT | 189 |
| V-type probe | CGCTAATAGTCGATAAAAAAAAAAATGACACACTGGGTGG | 190 |
| 7 + 8 − 1 − 5 | G | |
| V-type probe | CGCTAATAGTCGATCAAAAAAAAAATGACACACTGGGTGG | 191 |
| 7 + 7 − 1 − 5 | G | |
| V-type probe | GCTAATAGTCGATCAAAAAAAAAATGACACACTGGGTGGG | 192 |
| 6 + 7 − 1 − 5 | | |
| V-type probe | AGGAACTGGCTGCAGTAAAAAAAAAATGGCTACTACACTC | 193 |
| 8 + 7 − 2 − 5 | TT | |

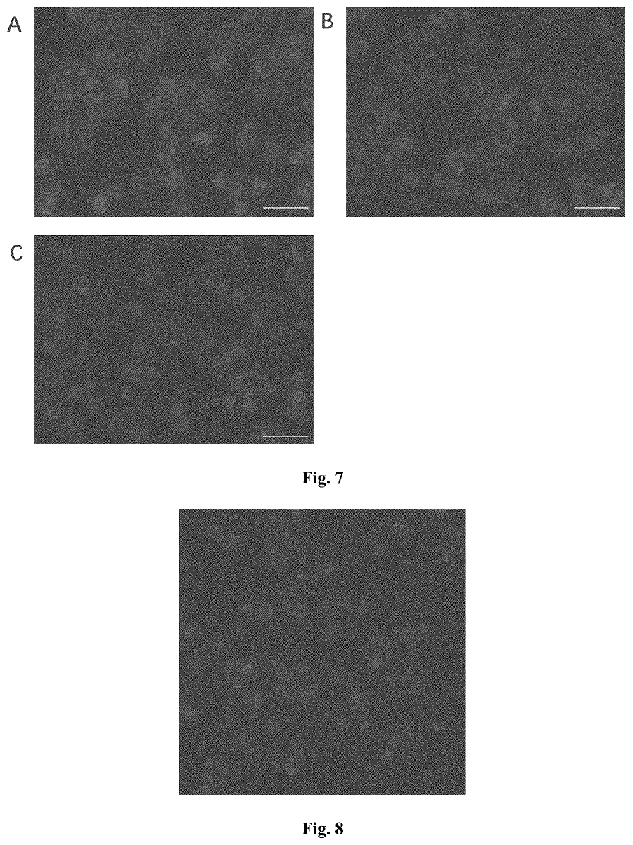

The experimental results were shown in FIG. 7 and Table 20. In the case that the V-type probe 2 had hybridization of 8 bp with the backbone sequence of the padlock probe and hybridization of 7 bp with the detection probe sequence of the padlock probe, when the V-type probe 1 had hybridization of 7 bp with the backbone sequence of the padlock probe and hybridization of 8 bp with the detection probe of the padlock probe, the extracellular noise was lower and the overall result was the most ideal.

TABLE 20
Number of fluorescent signals

SKRB3

| | HER2 | Cells |
|---|---|---|
| V1-5′6 + 7 | 17.91 | 435.00 |
| V1-5′7 + 7 | 46.92 | 445.00 |
| V1-5′7 + 8 | 62.38 | 474.00 |

### Example 6: Detection of Homopolar Double-C Probes

In this embodiment, homopolar double-C probes were designed to perform in-situ detection experiment of HER2 gene on SKBR3 cell line. The detection principle of the probes was shown in FIG. 10. The experimental steps were the same as those described in Example 1.1, and the specific probes and reagents used were shown in Tables 21 to 26 below.

TABLE 21
Probe sequences

| Description | Sequence | SEQ ID NO: |
|---|---|---|
| HER2 detection | AGTAGCCGTGACTATCGACT | 1 |
| probe sequence | | |
| HER2 padlock | ATTAGCGGTCCGTCTAGGAGAGTAGTACAGCAGCCGTCAAGAGTG | 2 |
| probe | TAGTAGCCGTGACTATCGACT | |
| HER2 RNA site 1 | CTCACCTACCTGCCCACCAATGCCAGCCTGTCCTTCCTGC | 19 |
| HER2 double-C | TTGGTGGGCAGGTAGGTGAGAAAAAAAAAACGCTAATAGTCGATA | 194 |
| probe 1-1 | | |
| HER2 double-C | GCAGGAAGGACAGGCTGGCAAAAAAAAAAATGGCTACTACACTCT | 21 |
| probe 2-1 | T | |
| HER2 RNA site 2 | TCGAAGCCTCACAGAGATCTTGAAAGGAGGGGTCTTGATC | 22 |
| HER2 double-C | AGATCTCTGTGAGGCTTCGAAAAAAAAAAACGCTAATAGTCGATA | 195 |
| probe 1-2 | | |
| HER2 double-C | GATCAAGACCCCTCCTTTCAAAAAAAAAAATGGCTACTACACTCTT | 24 |
| probe 2-2 | | |
| HER2 RNA site 3 | ACCTTTCTACGGACGTGGGATCCTGCACCCTCGTCTGCCC | 25 |
| HER2 double-C | TCCCACGTCCGTAGAAAGGTAAAAAAAAAACGCTAATAGTCGATA | 196 |
| probe 1-3 | | |
| HER2 double-C | GGGCAGACGAGGGTGCAGGAAAAAAAAAAATGGCTACTACACTCT | 27 |
| probe 2-3 | T | |

TABLE 22
Hybridization of probes

| C probes hybridization | stock | | final | | 1 x | |
|---|---|---|---|---|---|---|
| DEPC H₂O | | | | | 21.25 | ul |
| 2x Hyb buffer1 (12x SSC, 20% | 2 | x | 1 | x | 25 | ul |
| formamide) | | | | | | |
| HER2 double-C probe 1(3targets) | 2 | uM | 0.05 | uM | 1.25 | ul |
| HER2 double-C probe 2(3targets) | 2 | uM | 0.1 | uM | 2.5 | ul |
| Total | | | | | 50 | ul |

TABLE 23
Hybridization of padlock probe

| Circle-Bridge (PLP) hybridization | stock | | final | | 1 x | |
|---|---|---|---|---|---|---|
| DEPC H₂O | | | | | 28.75 | ul |
| T4 DNA ligase buffer(Thermo) | 10 | x | 1 | x | 5 | ul |
| 2.5M NaCl in 2% Tween-20 | 10 | x | 1 | x | 5 | ul |
| HER2 padlock probe | 2 | uM | 0.2 | uM | 5 | ul |
| BSA | 2 | ug/ul | 0.2 | ug/ul | 5 | ul |
| RiboLock RNase Inhibitor | 40 | U/ul | 1 | U/ul | 1.25 | ul |
| Total | | | | | 50 | ul |

TABLE 24
Ligation of padlock probe

| Ligation (Circularization) | stock | | final | | 1 x | |
|---|---|---|---|---|---|---|
| DEPC H₂O | | | | | 27.5 | ul |
| T4 DNA ligase buffer(Thermo) | 10 | x | 1 | x | 5 | ul |
| 2.5M NaCl in 2% Tween-20 | 10 | x | 1 | x | 5 | ul |
| ATP | 10 | mM | 1 | mM | 5 | ul |
| BSA | 2 | ug/ul | 0.2 | ug/ul | 5 | ul |
| 0.5 U/ul T4 DNA | 0.5 | U/ul | 0.0125 | U/ul | 1.25 | ul |
| ligase(Thermo) in 50% | | | | | | |
| Glycerol | | | | | | |
| RiboLock RNase Inhibitor | 40 | U/ul | 1 | U/ul | 1.25 | ul |
| Total | | | | | 50 | ul |

TABLE 25
Rolling circle amplification

| RCA | stock | | final | | 1 x | |
|---|---|---|---|---|---|---|
| DEPC H₂O | | | | | 26.75 | ul |
| Φ29 buffer | 10 | x | 1 | x | 5 | ul |
| Glycerol | 50% | | 5% | | 5 | ul |
| dNTP | 25 | mM | 1 | mM | 2 | ul |
| BSA | 2 | ug/ul | 0.2 | ug/ul | 5 | ul |
| Φ29 polymerase (Thermo) | 10 | u/ul | 1 | u/ul | 5 | ul |
| RiboLock RNase Inhibitor | 40 | u/ul | 1 | u/ul | 1.25 | ul |
| Total | | | | | 50 | ul |

TABLE 26
Hybridization of detection probe

| Detection | stock | | final | | 1 x | |
|---|---|---|---|---|---|---|
| H2O | | | | | 22.5 | ul |
| 2x Hyb buffer2 (4x SSC, 40% formamide) | 2 | x | 1 | x | 25 | ul |
| HER2 detection probe sequence | 2 | uM | 0.1 | uM | 2.5 | ul |

The microscopic examination results obtained in this experiment were shown in FIG. 8, which showed that when the V-type probe used in this method was deformed into a homopolar double-C probe, it could still work normally, and its signal detection amount was 37.61 under the current experimental scheme. Therefore, the homopolar double-C probe could be used as an alternative to the V-type probe in this method.

### Example 7: In-Situ Detection Experiment of V-Type Probe

In order to explore the broad applicability of the method of the present application on tissue samples and the role of the padlock probe, this example used the designed V-type probes to conduct in-situ detection experiments of UBC RNA on human liver tissue. The detection principle of the probes was shown in FIG. 9.

The specific experimental steps and reagents used were the same as those described in Example 1.1. The probes and sequences used in the experiments were shown in Table 27. The only difference from the experimental process of Example 1.1 was that two experiments were performed in the step of hybridizing the padlock probe. The experimental group was added with the padlock probe, the control group was not added with the padlock probe, and the other reagents were the same.

Wherein, the steps for processing human liver tissue samples comprised: human liver tissue was deforested within 10 minutes and fixed with 4% PFA for 5 minutes; rinsed twice with DEPC-PBS, 2 minutes each time; subjected to gradient dilution with 70%, 85% and 99.5% ethanol, 1 minute each time; and air-dried. An immunohistochemistry pen (ImmEdge Pen, purchased from VECTOR, Cat No. H-4000) was used to draw a hydrophobic circle around the tissue area to define the reaction area. After the hydrophobic circle was completely formed, washing was performed 3 times with DEPC-PBS-Tween. Permeabilization was carried out with 0.1M HCl for 5 minutes, and washing was performed 3 times with DEPC-PBS-Tween. The tissue sample prepared above was passed through 70%, 85%, and 100% gradient alcohol solutions successively at room temperature in the dark, and dehydration was carried out at each concentration for 2 minutes. After being naturally air-dried, the slides were sealed with anti-fluorescence quenching agent (SlowFade Gold Antifade Mountant, purchased from Invitrogen, Cat. No. S36936) containing DAPI (DAPI could penetrate the cell membrane to stain the nucleus, and emit blue fluorescence under detection conditions). Microscopic imaging was performed using a fluorescence microscope.

TABLE 27
Probe sequences

| Description | Sequence | SEQ ID NO: |
|---|---|---|
| UBC detection | TGCGTCTATTTAGTGGAGCC | 64 |
| probe | | |
| UBC padlock | ATTAGCGGTCCGTCTAGGAGAGTAGTACAGCAGCCGTCAAGAGTGTT | 65 |
| probe | GCGTCTATTTAGTGGAGCC | |
| RNA site 1 | CAGCCGGGATTTGGGTCGCAGTTCTTGTTTGTGGATCGCT | 34 |
| V-type probe 1-1 | CGCTAATGGCTCCACAAAAAAAAAATGCGACCCAAATCCCGGCTG | 35 |
| V-type probe 2-1 | AGCGATCCACAAACAAGAACAAAAAAAAAACAGACGCAACACTCTT | 36 |
| RNA site 2 | GGGATGCAGATCTTCGTGAAGACCCTGACTGGTAAGACCA | 37 |
| V-type probe 1-2 | CGCTAATGGCTCCACAAAAAAAAAATTCACGAAGATCTGCATCCC | 38 |
| V-type probe 2-2 | TGGTCTTACCAGTCAGGGTCAAAAAAAAAACAGACGCAACACTCTT | 39 |
| RNA site 3 | CAGAAAGAGTCCACTCTGCACTTGGTCCTGCGCTTGAGGG | 40 |
| V-type probe 1-3 | CGCTAATGGCTCCACAAAAAAAAAATGCAGAGTGGACTCTTTCTG | 41 |
| V-type probe 2-3 | CCCTCAAGCGCAGGACCAAGAAAAAAAAAACAGACGCAACACTCTT | 42 |
| RNA site 4 | TGGGCGCACCCTGTCTGACTACAACATCCAGAAAGAGTCC | 43 |
| V-type probe 1-4 | CGCTAATGGCTCCACAAAAAAAAAAAGTCAGACAGGGTGCGCCCA | 44 |
| V-type probe 2-4 | GGACTCTTTCTGGATGTTGTAAAAAAAAAACAGACGCAACACTCTT | 45 |
| RNA site 5 | GTGAAGACACTCACTGGCAAGACCATCACCCTTGAGGTCG | 46 |
| V-type probe 1-5 | CGCTAATGGCTCCACAAAAAAAAAATTGCCAGTGAGTGTCTTCAC | 47 |
| V-type probe 2-5 | CGACCTCAAGGGTGATGGTCAAAAAAAAAACAGACGCAACACTCTT | 48 |

The results were shown in FIG. 11. In the detection results, when the padlock probe was added (FIG. 11A), UBC had a large number of signals detected (white signal points) in the liver tissue, while there was no obvious signal detected when the padlock probe was not added (FIG. 11B).

This experiment showed that all signal sources generated by the method of the present application were based on the addition of the padlock probe, and no signal was detected in the experimental group without the addition of the padlock probe, which verified the reliability of the signal sources of the method of the present application. In addition, this experiment also illustrated that the method of the present application had wide applicability on tissue samples, and had the same high detection efficiency as on cell samples.

Although the specific embodiments of the present invention have been described in detail, those skilled in the art will understand that various modifications and changes can be made to the details based on all teachings that have been published, and these changes are within the protection scope of the present invention.. The full scope of the present invention is given by the appended claims and any equivalents thereof.

1. A method for detecting the presence or levels of one or more target nucleic acids in a sample, the method comprising:
(a) providing a detection sample suspected of containing one or more target nucleic acids, and, for each target nucleic acid, providing at least one probe set, wherein the probe set comprises a first probe, a second probe, a padlock probe and a detection probe;
wherein, the first probe has a sequence comprising: (i) a first complementary sequence that specifically binds to the padlock probe; (ii) a first target-binding sequence that specifically binds to the target nucleic acid; (iii) optionally, a first linker sequence for linking the first complementary sequence and the first target-binding sequence;
the second probe has a sequence comprising in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence;
the padlock probe is a single-stranded nucleic acid, which comprises: (i) a backbone sequence, and (ii) a detection probe sequence; under a condition that allows hybridization or annealing, the padlock probe is capable of hybridizing with or annealing to the first complementary sequence of the first probe and the second complementary sequence of the second probe to form a circular polynucleotide with a nick;
the detection probe comprises a detectable label and the detection probe sequence or fragment thereof;
(b) contacting the detection sample with the first probe, the second probe, the padlock probe, and a ligase under a condition that allows the ligase to ligate a nucleic acid nick;
(c) performing rolling circle amplification of the product of step (b) by using an amplification enzyme under a condition that allows the amplification;
(d) contacting the product of the previous step with the detection probe under a condition that allows hybridization or annealing, and detecting a signal from the detection probe bound to the product;
(e) detecting the presence or level of the target nucleic acid in the detection sample based on the presence or level of the signal from the detection probe.

2-10. (canceled)

11. The method according to claim 1, wherein the method has one or more characteristics selected from the following:
(1) for each target nucleic acid, providing at least 2, at least 3, at least 5, or more probe sets;
(2) the first linker sequence does not bind to the target nucleic acid or the padlock probe;
(3) the first target-binding sequence is located upstream or downstream of the first complementary sequence; and,
(4) the second linker sequence does not bind to the target nucleic acid or the padlock probe.

12. The method according to claim 1, wherein the method has one or more characteristics selected from the following:
(1) the first complementary sequence of the first probe hybridizes to a first region of the padlock probe, and the second complementary sequence of the second probe hybridizes to a second region of the padlock probe, and there is a spacer sequence between the first region and the second region; and,
(2) the first target-binding sequence and the second target-binding sequence are separated by 0 to 30 nt on the target nucleic acid.

13. The method according to claim 12, wherein the method has one or more characteristics selected from the following:
(1) the spacer sequence has a length of 0 to 30 nt;
(2) the spacer sequence has a length of 0 to 5 nt, 5 to 10 nt, 10 to 15 nt, 15 to 20 nt, 20 to 25 nt or 25 to 30 nt;
(3) the spacer sequence has a length of 0 nt, 3 nt, 5 nt, 8 nt or 10 nt;
(4) the first target-binding sequence and the second target-binding sequence are separated by 0 to 5 nt, 5 to 10 nt, 10 to 15 nt, 15 to 20 nt, 20 to 25 nt or 25 to 30 nt on the target nucleic acid; and,
(5) the first target-binding sequence and the second target-binding sequence are separated by 0 nt, 3 nt, 5 nt, 8 nt or 10 nt on the target nucleic acid.

14. The method according to claim 1, wherein the method has one or more characteristics selected from the following:
(1) the detection sample is selected from the group consisting of single cell, cell group, tissue, organ, or any combination thereof;
(2) the cell is selected from the group consisting of eukaryotic cell, prokaryotic cell, archaebacterial cell, artificial cell, or any combination thereof;
(3) the target nucleic acid is DNA and/or RNA;
(4) the detectable label is selected from the group consisting of fluorescent label, bioluminescent label, chemiluminescent label, isotope label, or any combination thereof; and,
(5) the amplification enzyme is a nucleic acid polymerase.

15. The method according to claim 14, wherein the method has one or more characteristics selected from the following:
(1) the fluorescent label is a fluorophore;
(2) the fluorescent label is selected from ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705, or any combination thereof;
(3) the nucleic acid polymerase is a DNA polymerase;
(4) the nucleic acid polymerase is a thermostable DNA polymerase;
(5) the nucleic acid polymerase is obtained from, Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus and Aquifex aeolieus; and,
(6) nucleic acid polymerase is Φ29 polymerase.

16. The method according to claim 1, wherein the method has one or more characteristics selected from the following:
(1) the detection sample is subjected to pretreatment; and
(2) the detection sample suspected of containing one or more target nucleic acids, the first probe, the second probe, the padlock probe and the ligase are provided, and the detection sample is allowed to contact with the first probe, the second probe, the padlock probe and the ligase, and then the detection probe is provided; or, the detection sample suspected of containing one or more target nucleic acids, the first probe, the second probe, the padlock probe, the ligase and the detection probe are provided, and the detection sample is allowed to contact with them.

17. The method according to claim 16, wherein the method has one or more characteristics selected from the following:
(1) the ligase is selected from the group consisting of T4 DNA ligase, DNA ligase I, DNA ligase III and DNA ligase IV; and,
(2) the pretreatment is selected from the group consisting of cell permeabilization, nucleic acid extraction, nucleic acid purification, and nucleic acid enrichment.

18. The method according to claim 1, wherein the first probe and the second probe have one or more characteristics selected from the following:
(1) the first probe and the second probe each independently comprise or consist of naturally occurring nucleotides, modified nucleotides, non-natural nucleotides, or any combination thereof;
(2) the first probe and the second probe each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt or 900 to 1000 nt;
(3) the first complementary sequence and the second complementary sequence each independently have a length of 10 to 15 nt, 15 to 20 nt, 20 to 30 nt, 30 to 40 nt, 40 to 50 nt;
(4) the first complementary sequence has a first portion complementary to the backbone sequence and a second portion complementary to the detection probe sequence;
(5) the second complementary sequence has a third portion complementary to the backbone sequence and a fourth portion complementary to the detection probe sequence;
(6) the first and second linker sequences each independently have a length of 5 to 10 nt, 10 to 15 nt, 15 to 20 nt, 20 to 30 nt, 30 to 40 nt, 40 to 50 nt; and,
(7) the first and second target-binding sequences each independently have a length of 12 to 15 nt, 15 to 20 nt, 20 to 30 nt, 30 to 40 nt, 40 to 50 nt.

19. The method according to claim 18, wherein the method has one or more characteristics selected from the following:
(1) the naturally occurring nucleotides are deoxyribonucleotides or ribonucleotides;
(2) the non-natural nucleotides are peptide nucleic acids (PNA) or locked nucleic acids;
(3) the first complementary sequence and the second complementary sequence each independently have a length of 10 to 20 nt;
(4) the first portion, the second portion, the third portion and the fourth portion each independently have a length of 0 nt to 15 nt;
(5) the first portion, the second portion, the third portion and the fourth portion each independently have a length of 5 nt, 6 nt, 7 nt, 8 nt, 9 nt or 10 nt;
(6) the first and second linker sequences each independently have a length of 5 nt, 6 nt, 7 nt, 8 nt, 9 nt, 10 nt, 11 nt, 12 nt, 13 nt, 14 nt or 15 nt; and,
(7) the first and the second target-binding sequences each independently have a length of 12 to 30 nt.

20. The method according to claim 1, wherein the detection probe has one or more characteristics selected from the following:
(1) the detection probes each independently comprise of naturally occurring nucleotides, modified nucleotides, non-natural nucleotides, or any combination thereof;
(2) the detection probes each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, or 900 to 1000 nt;
(3) the detection probes each independently have a 3′-OH terminus; or, the 3′-terminus of the probe is blocked;
(4) the detection probes are each independently linear or have a hairpin structure;
(5) the detection probes each independently bear a detectable label; and,
(6) the detection probe cannot be degraded by a nucleic acid polymerase.

21. The method according to claim 20, wherein the method has one or more characteristics selected from the following:
(1) the naturally occurring nucleotides is deoxyribonucleotides or ribonucleotides;
(2) the non-natural nucleotides is peptide nucleic acids (PNA) or locked nucleic acids;
(3) the 3′-terminus of the detection probe is blocked by adding a biotin or alkyl to the 3′-OH of the last nucleotide of the probe, or by removing the 3′-OH of the last nucleotide of the probe, or by replacing the last nucleotide with a dideoxynucleotide; and,
(4) the detection probes in the different probe sets bear different detectable labels.

22. The method according to claim 1, wherein the padlock probe has one or more characteristics selected from the group consisting of:
(1) the padlock probe is a linear continuous polynucleotide in its natural state;
(2) the padlock probe is a cyclic polynucleotide with a nick when hybridized or annealed to the first probe and the second probe;
(3) the padlock probes each independently comprise or consist of naturally occurring nucleotides, modified nucleotides, non-natural nucleotides, or any combination thereof;
(4) the padlock probes each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, or 900 to 1000 nt; and,
(5) the padlock probe cannot be degraded by a nucleic acid polymerase.

23. A probe set, wherein the probe set comprises a first probe, a second probe, a padlock probe and a detection probe, wherein:
the first probe comprises a sequence comprising: (i) a first complementary sequence that specifically binds to the padlock probe; (ii) a first target-binding sequence that specifically binds to a target nucleic acid; (iii) optionally, a first linker sequence for linking the first complementary sequence and the first target-binding sequence;
the second probe has a sequence comprising in the 5′ to 3′ direction: (i) a second target-binding sequence that specifically binds to the target nucleic acid; (ii) a second complementary sequence that specifically binds to the padlock probe; (iii) optionally, a second linker sequence for linking the second target-binding sequence and the second complementary sequence;
the padlock probe is a single-stranded nucleic acid, which comprises: (i) a backbone sequence, and (ii) a detection probe sequence; under a condition that allows hybridization or annealing, the padlock probe is capable of hybridizing or annealing to the first complementary sequence of the first probe and the second complementary sequence of the second probe to form a circular polynucleotide with a nick; and
the detection probe comprises a detectable label and the detection probe sequence or fragment thereof.

24. The probe set according to claim 23, wherein the probe set has one or more characteristics selected from the following:
(1) the first linker sequence does not bind to the target nucleic acid or the padlock probe;
(2) the first target-binding sequence is located upstream or downstream of the first complementary sequence; and,
(3) the second linker sequence does not bind to the target nucleic acid or the padlock probe.

25. A kit, comprising one or more probe sets according to claim 24.

26. The kit according to claim 25, wherein the kit further comprises a ligase, an amplification enzyme, a reagent for nucleic acid amplification, a reagent for rolling circle amplification, a reagent for detecting a fluorescent signal, or any combination thereof.

27. The kit according to claim 26, the kit has one or more characteristics selected from:
(1) the ligase is selected from the group consisting of T4 DNA ligase, DNA ligase I, DNA ligase III and DNA ligase IV;
(2) the amplification enzyme is a nucleic acid polymerase;
(3) the reagent for nucleic acid amplification comprises: working buffer for enzyme, dNTPs, water, solution containing ions, single-stranded DNA-binding protein, or any combination thereof; and,
(4) the reagent for rolling circle amplification is selected from the group consisting of RNase-free water, dNTPs, RNase inhibitor, or any combination thereof.

28. The kit according to claim 27, the kit has one or more characteristics selected from:
(1) the nucleic acid polymerase is a DNA polymerase;
(2) the nucleic acid polymerase is a thermostable DNA polymerase;
(3) the nucleic acid polymerase is obtained from, Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophilus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus and Aquifex aeolieus;
(4) nucleic acid polymerase is @29 polymerase; and,
(5) the kit is used to detect the presence or levels of one or more target nucleic acids in a sample.

29. The kit according to claim 25, wherein the kit has one or more characteristics selected from the following:
(1) the detection probes each independently comprise or consist of naturally occurring nucleotides, modified nucleotides, non-natural nucleotides, or any combination thereof;
(2) the detection probes each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, or 900 to 1000 nt;
(3) the detection probes each independently have a 3′-OH terminus; or, the 3′-terminus of the probe is blocked;
(4) the detection probes are each independently linear or have a hairpin structure;
(5) the detection probes each independently bear a detectable label;
(6) the detection probe cannot be degraded by a nucleic acid polymerase; and,
(7) the detection probes in the different probe sets bear different detectable labels.
